SEQUENCE LISTING (17 sequences)

SEQ ID NO: 1 - protein, 473 aa, Rattus norvegicus
MKRASSGGSRLLAWVLWLQAWRVATPCPGACVCYNEPKVTTSCPQQGLQAVPTGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSEVPCNLPQRLAGRDLKRLAASDLEGCAVASGPFRPFQTNQLTDEELLGLPKCCQPDAADKASVLEPGRPASAGNALKGRVPPGDTPPGNGSGPRHINDSPFGTLPGSAEPPLTALRPGGSEPPGLPTTGPRRRPGCSRKNRTRSHCRLGQAGSGSSGTGDAEGSGALPALACSLAPLGLALVLWTVLGPC

SEQ ID NO: 2 - protein, 285 aa, Rattus norvegicus
CPGACVCYNEPKVTTSCPQQGLQAVPTGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSEVPCNLPQRLAGRDLKRLAASDLEGCAV

SEQ ID NO: 3 - protein, 97 aa, Rattus norvegicus
PASAGNALKGRVPPGDTPPGNGSGPRHINDSPFGTLPGSAEPPLTALRPGGSEPPGLPTTGPRRRPGCSRKNRTRSHCRLGQAGSGSSGTGDAEGSG

SEQ ID NO: 4 - protein, 420 aa, Rattus norvegicus
MLPGLRRLLQGPASACLLLTLLALPPVTPSCPMLCTCYSSPPTVSCQANNFSSVPLSLPPSTQRLFLQNNLIRSLRPGTFGPNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFHGLSRLTILYLFNNSLASLPGEALADLPALEFLRLNANPWACDCRARPLWAWFQRARVSSSDVTCATPPERQGRDLRTLRDTDFQACPPPTPTRPGSRARGNSSSNHLYGVAEAGAPPADPSTLYRDLPAEDSRGRQGGDAPTEDDYWGGYGGEDQRGEQTCPGAACQAPADSRGPVLSAGLRTPLLCLLLLAPHHL

SEQ ID NO: 5 - protein, 11 aa, Rattus norvegicus
PTRPGSRARGN

SEQ ID NO: 6 - protein, 13 aa, Rattus norvegicus
PTRPGSRARGNSS

SEQ ID NO: 7 - protein, 17 aa, Rattus norvegicus
TGPRRRPGCSRKNRTRL

SEQ ID NO: 8 - protein, 96 aa, Rattus norvegicus
HINDSPFGTLPGSAEPPLTALRPGGSEPPGLPTTGPRRRPGCSRKNRTRSHCRLGQAGSGSSGTGDAEGSGALPALACSLAPLGLALVLWTVLGPC

SEQ ID NO: 9 - protein, 125 aa, Rattus norvegicus
PGRPASAGNALKGRVPPGDTPPGNGSGPRHINDSPFGTLPGSAEPPLTALRPGGSEPPGLPTTGPRRRPGCSRKNRTRSHCRLGQAGSGSSGTGDAEGSGALPALACSLAPLGLALVLWTVLGPC

SEQ ID NO: 10 - protein, 160 aa, Rattus norvegicus
GPFRPFQTNQLTDEELLGLPKCCQPDAADKASVLEPGRPASAGNALKGRVPPGDTPPGNGSGPRHINDSPFGTLPGSAEPPLTALRPGGSEPPGLPTTGPRRRPGCSRKNRTRSHCRLGQAGSGSSGTGDAEGSGALPALACSLAPLGLALVLWTVLGPC

SEQ ID NO: 11 - protein, 473 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 12 - protein, 473 aa, Mus musculus
MKRASSGGSRLLAWVLWLQAWRVATPCPGACVCYNEPKVTTSCPQQGLQAVPTGIPASSQRIFLHGNRISHVPAASFQSCRNLTILWLHSNALARIDAAAFTGLTLLEQLDLSDNAQLHVVDPTTFHGLGHLHTLHLDRCGLRELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLMPLRSLQYLRLNDNPWVCDCRARPLWAWLQKFRGSSSEVPCNLPQRLADRDLKRLAASDLEGCAVASGPFRPIQTSQLTDEELLSLPKCCQPDAADKASVLEPGRPASAGNALKGRVPPGDTPPGNGSGPRHINDSPFGTLPSSAEPPLTALRPGGSEPPGLPTTGPRRRPGCSRKNRTRSHCRLGQAGSGASGTGDAEGSGALPALACSLAPLGLALVLWTVLGPC

SEQ ID NO: 13 - protein, 478 aa, Danio rerio
MFKRGCGLEFLLVLCGLELSWSCPRHCICYMAPSTVSCQAHNFLSVPEGIPPHSERIFLQNNKIHRLLQGHFSPTTVTLWIYSNNITYIEPSTFQGFTLLEELDLGDNRYLRSLSAETFHGLGRLHALHLYRCGLSALPNNIFQGLRNLQYLYLQDNHLEYLQDDLFVDLHNLSHLFLHGNRLWSLHQNTFRGLGALDRLLLHHNQLQWVDRLAFHDLRRLTTLYLFNNSLTELAGECLTQLPALEYLRLNDNPWECDCKALSLWDWLKKFRGSTSSVGCVAPAELAGKDLKQLRKEDFPNCSGSESLHQSKTNTWAGTNKVSLKQEPHPAQPPQTHPHHPQLNEQFPSPPSPLPQPPPAINGVQVVPGVSPDMIPDQRPGRSRNCTRQRVRGSKGKGPNEVHILKEMADKEYSSPDFTGKYDETSSNGSNTRRKHKCTPRTSVRPPSGVQQATNLGHSHHTHLFLCSISGLLTLILR

SEQ ID NO: 14 - protein, 833 aa, Gallus gallus
MRSDVAKGPSKCVRGQPVAGVMDASHGRADVVSVGFTLEGCLSLSLQRKGIIASLVPVGSCSPGCQQRSSECCNAALLLDGLLQIHGQSFIHCCGPSLLSLSEPTFAVQGVPWAAQLGARCWEELTAVAQQWWDALPPRGMELMELLWDVTQPMLQEGAHSPAGLSIPGILTHSWPFPLLDEDVQSCQQSWPCNAFKAAPASHEMVPQRHRSYSVCALLPYTTDTSTFCSSDLVLGVLSIAPEHRSSLCLCLTASSSQAGGDGMSSCPCHGTAVCHSHPLVLQVFGTCASQQPVEIEHTCSAEPAEEWSPLWQTWSSSDPWRVCLHKGLTARHLHGAADVHQPQNLLLQLSARSLPEGSKLLILVLCLNIQSEVESCPGACVCYSEPKITISCQQQGLTAIPTEIPIQSQRIFLHNNKITLVRSTSFTSCRNMTILWIHSNNISLIEPGAFYGLNKLEELDLSDNTNLKSINPVTFRGLVHLHTLHLDRCGLMELSTGLFRGLFSLQYLYLQDNNLQNLLDDTFIDLANLTYLFLHGNKIKSLSENVFRGLINLDRLLLHQNRVSLVHRRSFHDLGKVMTLYLFNNNLTVLTGETMAPLVSLQYLRLNGNQWICDCQARSLWNWFKQFKGSSSELECHLPPHLAGRDLKRLQSSDLEGCIDSFNQIRTSVFSTKTRSGKLATGSPPLSSHDGSMKCCQPEMDKSFIYEAKGKAGPSSHSSRPSSNNPLKDKENMSKTKYVETDPSKNGSNKQINDSPFGTFPSIVDPPLTKLRPEFLEPIEPSTVPTKKRQGCSKKNKSKAQCRLTQQGNSSTLQLSLSLLIPPLVWSLLLLC

SEQ ID NO: 15 - protein, 420 aa, Mus musculus
MLPGLRRLLQGPASACLLLTLLALPSVTPSCPMLCTCYSSPPTVSCQANNFSSVPLSLPPSTQRLFLQNNLIRSLRPGTFGPNLLTLWLFSNNLSTIHPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFHGLSRLTILYLFNNSLASLPGEALADLPALEFLRLNANPWACDCRARPLWAWFQRARVSSSDVTCATPPERQGRDLRALRDSDFQACPPPTPTRPGSRARGNSSSNHLYGVAEAGAPPADPSTLYRDLPAEDSRGRQGGDAPTEDDYWGGYGGEDQRGEQTCPGAACQAPADSRGPALSAGLRTPLLCLLPLALHHL

SEQ ID NO: 16 - protein, 420 aa, Homo sapiens
MLPGLRRLLQAPASACLLLMLLALPLAAPSCPMLCTCYSSPPTVSCQANNFSSVPLSLPPSTQRLFLQNNLIRTLRPGTFGSNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFRGLSRLTILYLFNNSLASLPGEALADLPSLEFLRLNANPWACDCRARPLWAWFQRARVSSSDVTCATPPERQGRDLRALREADFQACPPAAPTRPGSRARGNSSSNHLYGVAEAGAPPADPSTLYRDLPAEDSRGRQGGDAPTEDDYWGGYGGEDQRGEQMCPGAACQAPPDSRGPALSAGLPSPLLCLLLLVPHHL

SEQ ID NO: 17 - protein, 411 aa, Tetraodon nigroviridis
MRNCKSGLSLWLLVWLVLGKPSLTSACPHLCVCYPTPMTVSCQAQNFTAVPAGVPYESQRVFLQNNRITELRVGSFGFGTQVLWLFSNNITWIEAGAFSELRDLEELDLGDNPGLRRLEGGAFRGLEKLQSLHMHRCRLSALPHDIFHKLYSLQFLYLQENHLHFLQDDIFSDLINLSQLFLHGNRIRTLSENVFRGLVNLDRLLLHDNRIRQVNRRAFRDLGRLTMLFLFNNSLAELPSQTLRDTQSIEFLRLNANPWACGCESRALWEWFREARVSSSEVICASPSTRRGQDLRFLREMDFALCPLPDPGVFEKTSETVKAGLYGKGPSTTSSVVKYELGEEELALPKLDPEEYWENYGNEDSGITVRCFELECPPEFDLPPSASSPSSASPSLVALSAASVLLHLLFG

(19) United States
(12) Patent Application Publication
(10) Pub. No.: US 2010/0022453 A1
(43) Pub. Date: Jan. 28, 2010

(54) NoGo Receptor 1 and Fibroblast Growth Factor Interactions

(75) Inventors: Roman Giger, Rochester, NY (US); Hakjoo Lee, Pittsford, NY (US); Steven Raiker, Rochester, NY (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 12/439,373

(22) PCT Filed: Aug. 30, 2007
(86) PCT No.: PCT/US07/77252

§ 371 Date: Jun. 18, 2009

Related U.S. Application Data
(60) Provisional application No. 60/823,946, filed on Aug. 30, 2006.

(51) Int. Cl. A61K 38/16; C07K 14/435; C07H 21/04; C12N 15/63; C12N 5/10; C12N 5/02; A61K 31/7088; A61P 25/00

(52) U.S. Cl. 514/12; 530/350; 536/23.5; 435/320.1; 435/325; 536/23.4; 435/375; 514/44 A

(57) ABSTRACT

Compositions and methods useful in promoting neuronal growth, synaptic transmission, or neuronal regeneration are described, including, for example, a polypeptide comprising a fragment of NgR1, wherein the NgR1 fragment has reduced FGF2 binding as compared to wild-type NgR1. Also described are chimeric polypeptides comprising the NgR1 fragment and compositions comprising the fragment or chimeric polypeptide. Nucleic acids, vectors and expression systems are also described which encode the fragments and polypeptides. These compositions can be used in combination with FGF2 to promote neurite outgrowth or neuronal regeneration and can be used to treat central nervous systems diseases and disorders.

### CROSS-REFERENCE TO RELATED APPLICATIONS

This application claims the benefit of U.S. Provisional Application No. 60/823,946, filed Aug. 30, 2006, which is incorporated by reference herein in its entirety.

### STATEMENT REGARDING FEDERALLY SPONSORED RESEARCH

This invention was funded by the National Institute of Neurological Disorders and Stroke (Grant No. RO1 NS047333-01). Therefore, the United States Government may have certain rights in this invention.

### BACKGROUND

In the mature nervous system, neuronal growth and structural plasticity are regulated by a complex interaction of growth promoting and growth inhibitory extracellular cues, and alteration of neuronal structure has been directly linked to change in synaptic transmission. Although various cues have been identified that either promote or inhibit growth and plasticity of mature neurons, lacking in the field is an understanding of how to modulate promoters and inhibitors concurrently in the complex milieu of the central nervous system. To date, interactions between the various promoters and inhibitors of growth and means of influencing these interactions were poorly understood.

### SUMMARY

The Nogo receptor NgR1 binds with high affinity to the myelin inhibitory constituents NogoA, MAG, and OMgp. In addition, NgR1 supports high affinity binding of the fibroblast growth factor family members FGF1 and FGF2. Provided herein are compositions and methods for promoting neuronal growth or regeneration, for promoting synaptic strength and memory, for treating neurodegenerative diseases or conditions, and for promoting memory by inhibiting NgR1 expression or activity. For example, treatment for central nervous system injury and Alzheimer's Disease are described. Conversely, promoting NgR1 expression or activity is useful for reducing synaptic strength and for treating disorders associated with excitatory neurotransmission, such as a seizure disorder.

Thus, provided herein is a polypeptide comprising a fragment of NgR1, wherein the NgR1 fragment has reduced FGF2 binding as compared to wild-type NgR1. A soluble form of this polypeptide preferentially binds and sequesters myelin inhibitors but not FGF1 or FGF2. Also described are chimeric polypeptides comprising the fragment and compositions comprising the fragment or polypeptide. Nucleic acids, vectors and expression systems are also described which encode the fragments and polypeptides. These compositions can be used with FGF2 to promote neurite outgrowth, cell survival, neuronal regeneration, neuronal plasticity, synaptic strength, and memory and can be used to treat central nervous systems diseases and disorders.

Also, provided herein is a method of screening for a molecule that modulates (i.e., increases or decreases) activity-dependent synaptic strength. Such a method comprises, for example, contacting a NgR1-containing cell with the agent to be tested, providing an agent that stimulates activity, and detecting a change in activity in the presence of the agent to be tested.

The details of one or more embodiments are set forth in the accompanying drawings and the description below. Other features, objects, and advantages will be apparent from the description and drawings, and from the claims.

### DESCRIPTION OF DRAWINGS

FIG. 1 shows a graph of the number of axonal branches (0 to more than 5) on axons of E18 rat cortical neurons transfected with NgR1 or GFP plasmid DNA and cultured in the absence or presence of FGF2 (25 ng/ml) (bFGF) for 2 days. Cells were fixed and stained with anti-NgR1 or TuJ1.

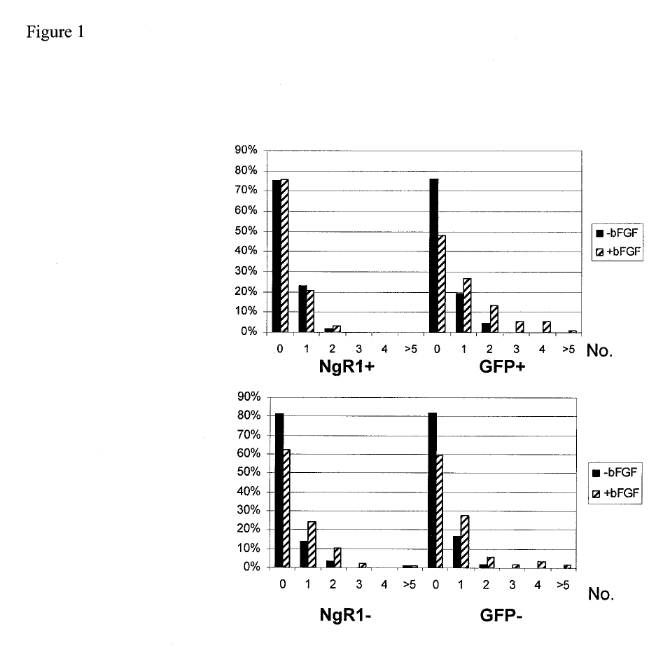

FIGS. 2A-C show micrographs indicating that NgR1 and NgR3 associate with neural glycans in E18 rat tissue. In situ binding of soluble AP-sNgR1 fusion-protein to neonatal (A and B) and E18 (C) rat brain tissue sections. AP-sNgR1 binds to many fiber systems, including the internal capsule (IC in A), thalamo-cortical projections (arrow in A), fimbria-fornix (star in B), hippocampal alveus (arrowhead in B), and optic nerve (C). FIGS. 2D-G show a comparison of binding of AP-sNgR1 (D), AP-sNgR2 (E), AP-sNgR3 (F), and AP only (G) to E18 brain tissue sections. FIG. 2H is a schematic showing the structural basis of soluble receptor binding to brain tissue sections. The C-terminal cysteine-rich cap (CT) and stalk domain of NgR1 and NgR3 are sufficient to support binding to brain tissue. FIG. 2I shows AP-fusion proteins used for tissue binding studies analyzed by anti-AP immunoblotting. FIG. 2J shows a histogram of quantification of relative binding strengths of soluble receptors to E18 brain tissue normalized to AP-sNgR1. FIGS. 2K and L show serial sections of E18 brains incubated with Heparinase III (Hep'ase) or no enzyme (control) prior to binding of AP-sNgR1 or AP-sNgR3. FIG. 2M is a histogram showing quantification of relative binding strengths of AP-sNgR1 following preincubation of brain sections with PBS (control), glycopeptidase F (GlyF), N-acetylglucosaminidase (NAC), Heparinase (Hep'ase), chondroitinase ABC (Ch'ase), V. cholerae neuraminidase (VCN), or endoneuraminidase N (EndoN). In the presence of heparin (100 Pg/ml), binding of AP-sNgR1 is completely blocked. Asterisks indicate significantly reduced binding compared to control, (p value<0.05). Student's t-test. Scale bar, A=200 μm; B=50 μm; C=25 μm, D-G, K and L=200 μm.

FIG. 3 shows that NgR1 supports binding of select members of the FGF family. FIG. 3A shows micrographs with NgR1 expressed on the cell surface of COS-7 supporting binding of AP-FGF1, FGF2, and to a lesser extent FGF4. No binding of FGF8, FGF9, or FGF21 to NgR1 was observed. NgR2 binds MAG-Fc but does not support binding of AP-tagged FGF fusion proteins. Scale bar, 20 μm. FIG. 3B shows pull-down experiments with NgR1-Fc and AP-fusion proteins, revealing a direct interaction of NgR1 with FGF2 and Nogo66, but not NiG, or VEGF. Excess IgG competes with NgR1-Fc for binding to protein A/G beads and blocks the pull down of AP-FGF2. FIG. 3C shows cross-linking of ¹²⁵I-FGF2 to NgR1-Fc and FGFR1-Fc in the presence or absence of excess “cold” FGF2 or insulin. Troy-Fc and ephrinB3-Fc were used as negative controls. Complexes of ¹²⁵I-FGF2:NgR1-Fc (˜210 kDa) and ¹²⁵I-FGF2:FGFR1-Fc (˜230 kDa) were resolved by 7% SDS-PAGE and the dried gel was exposed to X-ray film. The arrowhead denotes radiolabeled complexes. FIGS. 3D and E show Scatchard plot analysis of AP-FGF1 (D) and AP-FGF2 (E) binding to NgR1 expressing COS cells. The inserts show saturation binding curves.

FIG. 4 shows immunohistochemistry of high affinity binding of alkaline phosphatase tagged FGF2 upon deletion of various regions of NgR1 (deletion of the heparin binding motif within NgR1 (Δ29), deletion of 41 amino acids from the C-terminus of FGF2 (Δ41), deletion of the unique region (Δ unique), deletion of the LRRCT domain (Δ LRR-CT), and deletion of the leucine-rich repeat cluster (amino acids 1-310) (Δ LRR)).

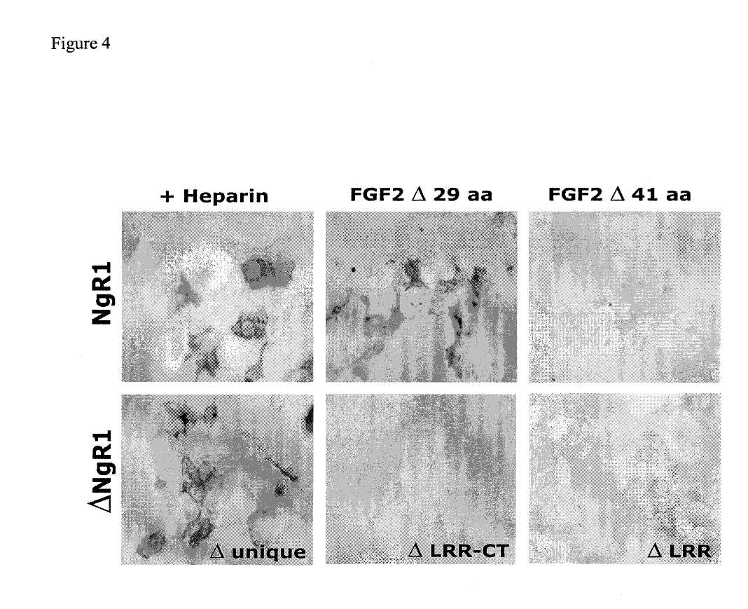

FIGS. 5A-G show that NgR1 suppresses FGF2-elicited differentiation of PC12 cells. FIG. 5A shows a mixed culture of control PC12 and PC12 NgR1 cells grown for four days in the presence of FGF2 and stained with anti-NgR1 (red) and anti-E-tubulin III (TuJ1) (green). The outgrowth of neurite-like processes is selectively attenuated in NgR1-positive PC12 cells. Scale bar, 100 μm. FIG. 5B shows a Western blot analysis of cell lysates of PC12 control cells and the PC12^{NgR1} cell lines NgR1-1 and NgR1-2. Actin is shown as a loading control. FIG. 5C shows a histogram indicating quantification of FGF2-elicited cell differentiation. In the presence of 25 ng/ml FGF2, significantly more PC12 cells show processes longer than two cell bodies in diameter compared to PC12^{NgR1} cells (asterisk indicates p<0.05, t-test). FIG. 5D shows Western blots indicating that increasing the concentration of FGF2 leads to an increasing strong activation of the MEK-ERK1/2 pathway in PC12 cells. In PC12^{NgR1} cells, however, FGF2-elicited activation of the MEK-ERK1/2 pathway is strongly inhibited. In the presence of EGF, dose-dependent activation of the MEK and ERK1/2 is observed in both PC12 and PC12^{NgR1} cells. FIG. 5E shows that, similar to FGF2, FGF1-elicited activation of ERK1/2 is strongly inhibited in PC12NgR1 cells. FIG. 5F shows that, in PC12^{NgR1} cells, FGF2-elicited activation of the adaptor protein FRS2α is strongly attenuated. Membrane fractions of control and PC12^{NgR1} cells treated with FGF2 or EGF were analyzed using anti-phospho-specific FRS2α (Tyr⁴³⁶) and anti-FRS2α antibodies. (C=control; F=FGF2; and E=EGF). FIG. 5G shows the results of rescue experiments in PC12^{NgR1} cells with constitutively active RasV14 (caRas), FGFR1 and FGFR3. Phosphorylation of ERK1/2 in PC12^{NgR1} cells is restored in FGFR1 or FGFR3 transfected cells in an FGF2-dependent manner.

FIGS. 6A-I show that FGF2 enhances hippocampal LTP in NgR1 mutants. FIGS. 6A-B show Niss1 staining of adult NgR1^{+/+}(A) and NgR1^{-/-} (B) hippocampal sections. FIG. 6C shows an in situ hybridization of NgR1 expression in the hippocampus. FIGS. 6D-I show the results of recording field excitatory postsynaptic potentials (fEPSP) at Schaffer collateral-CA1 synapses in acute hippocampal slices from wild-type (+/+) and NgR1 mutant (−/−) mice. FIG. 6D shows input-output curves for basal synaptic transmission, revealing no differences between NgR1+/+ and NgR1−/−slices. FIG. 6E shows LTP in NgR1^{-/-} and wild-type slices. fEPSPs were recorded at CA1 synapses and slopes were plotted against time before and after tetanic stimulation (two trains of stimuli at 100 Hz for 1 sec, separated by a 10 sec interval). FIG. 6F shows quantification of LTP at 40-45 minutes of NgR1^{+/+} and NgR1^{-/-} slices revealed no difference in fEPSP slope ratio. FIG. 6G shows that, in the presence of FGF2 locally applied via the recording electrode, NgR1^{-/-} slices have significantly enhanced LTP compared to NgR1^{+/+}. FIG. 6H shows that local application of FGF8, a FGF family member that does not bind to NgR1, does not result in enhanced LTP in NgR1^{-/-} slices. FIG. 6I shows quantification of LTP at 40-45 min in the presence of FGF2 and FGF8 in NgR1^{+/+} and NgR1^{-/-} slices. Representative traces before and after LTP are shown as inserts, calibration 0.5 mV, 5 msec. All values are mean±SEM. Asterisk indicates p<0.001. Scale bar, 200 μm.

FIGS. 7A-D show that NgR1 is localized to hippocampal synapses and short-term plasticity is unaltered in NgR1 mutants. FIG. 7A shows a time-course of NgR1 protein expression in rat hippocampus. Equal amounts (10 μg) of cell lysate between ages E18 to P70 were analyzed by anti-NgR1 immunoblotting. Actin is shown as a loading control. FIG. 7B shows the results using a synaptosomal membrane preparation of adult rat hippocampus. Equal amounts (10 μg) of protein from each fraction were probed with antibodies specific for NgR1, OMgp, FGFR1, syndecan-3, and the markers NR1 and PSD-95 (postsynaptic), syntaxin 1A (presynaptic), and synaptophysin (extra-synaptic junction). FIG. 7C shows PPF, the increase in the second fEPSP slope over the first, calculated in NgR1^{+/+} and NgR1^{-/-} slices, in the presence (+) or absence (−) of locally applied FGF2. Mean values were plotted against different interpulse intervals. FIG. 7D shows PTP, in the presence and absence of locally applied FGF2, in NgR1^{+/+} and NgR1^{-/-} hippocampal slices.

FIGS. 8A-C show FGFR kinase activity is necessary for FGF2-enhanced LTP in NgR1 mutants. FIG. 8A shows that in the presence of SU5402, FGF2-elicited enhancement of LTP in NgR1^{-/-} slices is suppressed. fEPSPs were recorded as described above. FIG. 8B shows quantification of LTP at 40-45 minutes in NgR1^{-/-} slices following local application of FGF2 revealed a significant reduction of the fEPSP slope ratio in the presence of SU5402. FIG. 8C shows dose-dependent inhibition of FGF2− but not EGF-elicited ERK1/2 activation in PC12 cells treated with SU5402. Cell lysates were analyzed by Western blotting with anti-phospho-ERK1/2 and normalized to actin.

FIGS. 9A-F show OMgp negatively regulates hippocampal LTP. FIG. 9A shows a recording of fEPSP at CA1 synapses in acute hippocampal slices from acute NgR1^{+/+} hippocampal slices locally treated with OMgp is significantly decreased compared to untreated NgR1^{+/+} slices. Representative traces before and after LTP are shown as inserts, calibration 0.5 mV, 5 msec. FIG. 9B shows LTP is unaltered in NgR1^{-/-} slices treated with OMgp compared to untreated NgR1^{-/-} slices (—OMgp). FIG. 9C shows quantification of LTP at 40-45 min in the presence of OMgp in NgR1^{+/+} and NgR1^{-/-} slices. All values are mean±SEM. Asterisk indicates p<0.001. FIG. 9D shows PPF induced at interpulse intervals of 25 to 500 ms was not significantly different in NgR1^{+/+} control (n=3 slices/2 animals) or OMgp treated slices (n=6 slices/3 animals p>0.05). FIGS. 9E and F show that PTP is not significantly different in NgR1^{+/+} between controls (n=3 slices/2 animals) and OMgp treatment (n=6 slices/2 animals, p>0.05).

FIGS. 10A-F show NgR1 mutant mice have altered distribution of dendritic spine morphologies. FIG. 10A shows dendrites of adult wild-type (NgR1^{+/+}) and mutant (NgR1^{-/}) mouse hippocampal CA1 pyramidal neurons stained by Golgi impregnation. FIG. 10B shows CA1 dendritic spines of NgR1^{+/+} and NgR1^{-/-} mice along apical dendrites. FIG. 10C shows quantification of spine morphologies: assigning individual spines into different classes (stubby, mushroom, and thin) revealed a significantly altered spine distribution profile in NgR1 mutants (n=12) compared to wild-type controls (n=8). Double asterisks, p<0.001; single asterisk, p<0.05. FIG. 10D is a schematic showing morphological categories to which spines were assigned. FIG. 10E is an ultrastructural analyses of synapse density in area CA1 of NgR1^{+/+} (559 synapses/n=2 animals) and NgR1^{-/-} (769 synapses/n=3 animals). FIG. 10F shows quantification of the results shown in E. Hippocampi revealed no significant changes (p=0.214) between the two genotypes. Scale bars, 25 μm(A), 5 μm (B), and 0.2 μm (E).

FIGS. 11A-D show that NgR1 forms a complex with the neural HSPG syndecan-3. FIG. 11A shows a FPLC ion-exchange chromatogram of P7 rat brain homogenates fractionated in a linear salt gradient. FIG. 11B shows FPLC fractions spotted on a nitrocellulose membrane, first assayed for supporting AP-sNgR1^{CT+stalk} binding (top) and then reprobed for immunoreactivity with anti-syndecan-3 (bottom). Strongest binding of AP-sNgR1^{CT+stalk} was supported by FPLC fractions 16-25, which closely overlapped with anti-Syn-3 immunoreactive fractions. FIG. 11C shows Syn-3 from P7 brains forming a complex with AP-sNgR1 but not with AP-sNgR2, as assessed by immunoprecipitation with anti-AP followed by anti-Syn-3 Western blotting. FIG. 11D shows serial sections of rat E18 brain, with AP-sNgR1 binding, anti-syndecan-3 immunolabeling, and AP-HBGAM binding. The syndecan-3 ligand AP-HBGAM and AP-sNgR1 show very similar binding patterns. Overlap between AP-sNgR1 and anti-syn3 labeling is seen in the internal capsule (IC) and along thalamo-cortical fibers, the developing hippocampus (arrowhead), and the habenula (asterisk). Of note, the marginal zone (double arrow) supports binding of AP-sNgR1 but is not immunoreactive for syndecan-3, suggesting that sNgR1 associates with additional binding partners.

FIGS. 12A-B show that NgR1 mediated suppression of the ERK-MAP kinase pathway is cell type specific. Bovine endothelial cells (GM7373) that stably expressing NgR1 were generated using Lipofectamine (Invitrogen, San Diego, Calif.), followed by selection with G418. FIG. 12A shows a Western blot analysis with anti-NgR1, showing NgR1 expression in GM^{NgR1-1} and GM^{NgR1-2} cell lines. NgR1 was not detectable in control GM7373 cells. FIG. 12B shows that in the presence of FGF2, GM7373 control cells show a robust and dose-dependent increase in ERK1/2 activation.

FIGS. 13A-F show control experiments for electrophysiological recordings. FIG. 13A shows tissue diffusion of molecules locally applied through the recording pipette. Texas red-conjugated dextran (MW 10 kDa; 10 mg/ml) was locally applied to the CA1 dendritic field in acute mouse hippocampal slices. Within a 30 minute time period, labeled dextran spreads radially from the point of application over more that 100 μm, as assessed by fluorescence microscopy. FIG. 13B shows suppression of LTP in wild-type hippocampal slices following HFS (100 Hz, 1 s duration, 2 trains, interval 10 s) in the absence (n=11 slices/9 animals) or the presence (n=4 slices/2 animals; p<0.01) of AP5, an NMDA receptor antagonist (100 μM in the recording pipette). Small inserts show traces, calibration 0.5 mV, 5 msec. FIG. 13C shows quantification of LTP shown in FIG. 13B at 45 min. FIGS. 13 D and E show induction of LTP in the presence of the FGFR kinase inhibitor, SU5402, locally applied via the recording electrode (1 mM; concentration in recording electrode), in NgR1^{+/+} (D, ACSF n=8 slices/7 animals; +SU5402 n=6 slices/4 animals) and NgR1^{-/-} slices (E, ACSF n=8 slices/8 animals; +SU5402 n=6 slices/3 animals). FIG. 13F shows quantification of LTP at 45 min shown in (D and E). LTP is not significantly altered in NgR1^{+/+} and NgR1^{-/-} slices in the presence of SU5402.

FIG. 14 shows graphs of the analysis of CA1 apical dendritic spine density classified by spine morphology of four littermate groups. Each group of wild-type (NgR1^{+/+}) and NgR1^{-/-} littermates (10 animals total) exhibited an increase in stubby-shaped spines and, decreases in mushroom- and thin-shaped spines. Each of the groups 1-4 represents littermates with wild-type (WT) and NgR1 mutant (null) mice.

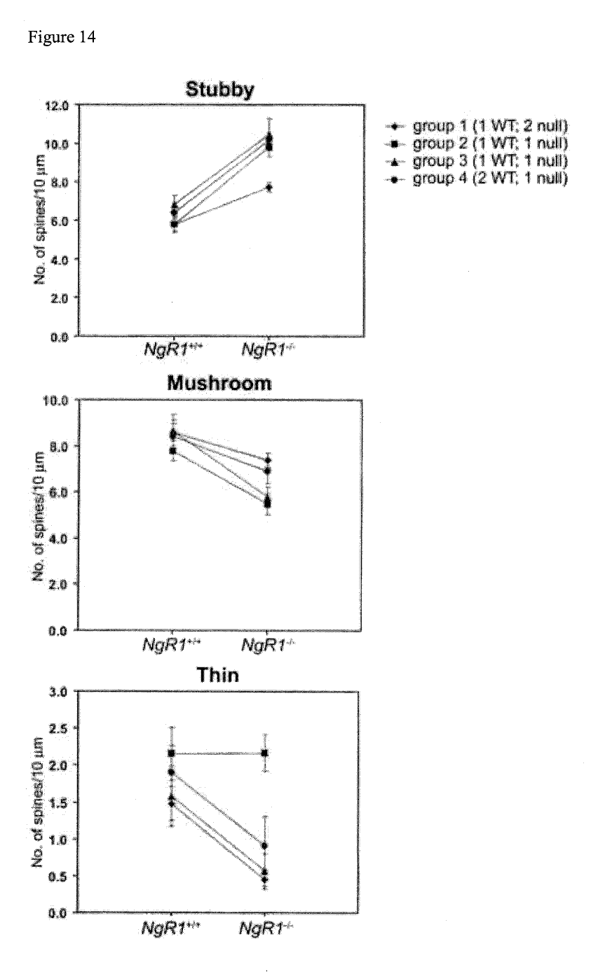

Like reference symbols in the various drawings indicate like elements.

### DETAILED DESCRIPTION

Growth factors stimulate neurons to grow and regenerate. Nogo-A, myelin-associated glycoprotein (MAG), and oligodendrocyte myelin glycoprotein (OMgp), however, have opposing effects and are potent myelin-derived inhibitors of neuronal growth and regeneration. One mechanism for each of these myelin-derived inhibitors to bring about inhibition is through association with the Nogo-66 receptor (NgR1), a lipid anchored neuronal cell surface glycoprotein. NgR1 is a member of a small family of Nogo receptors (NgRs), comprised of NgR1, NgR2, and NgR3. NgR-receptors are characterized by a cluster of eight canonical leucine-rich repeats (LRRs) flanked by cysteine-rich, N-terminal (LRRNT) and C-terminal (LRRCT) ‘cap’ domains. The LRR cluster is connected through a stalk (‘unique’ domain) to a GPI anchor for membrane attachment (Venkatesh et al., J. Neurosci. 25:808-22, 2005.)

NgR1 co-immunoprecipitates with syndecan-3, a neural heparin sulfate proteoglycan, which forms a complex with Fibroblast Growth Factor 2 (FGF2; also known as basic FGF or bFGF.). As shown herein, NgR1 directly interacts with FGF2 to suppress FGF2-mediated growth promoting pathways. Thus, NgR1 activates growth inhibitory pathways and in addition blocks growth promoting pathways.

There is a novel partnership between NgR1 and the FGF-FGFR receptor system. NgR1 is a high affinity receptor for select members of the FGF family that functions as a negative regulator of FGF2/FGFR signaling. First, ectopic NgR1 blocks FGF2-induced differentiation of PC12 cells. Second, ectopic expression of NgR1 in primary cortical neurons suppresses FGF2 elicited axonal branching. Third, loss of NgR1 in acute hippocampal slices results in an FGF2-dependent increase in long-term synaptic plasticity at the CA3-CA1 synapse. Consistent with the idea that NgR1 regulates functional synaptic plasticity, OMgp inhibits LTP at the CA3-CA1 synapse in an NgR1-dependent manner. Anatomical studies of NgR1 mutant brains revealed a dendritic spine phenotype along apical dendrites of hippocampal CA1 pyramidal neurons. Thus NgR1 ligands like OMgp have a role in regulating synaptic plasticity and establish a new function for NgR1 in regulating neuronal structure and ligand-dependent synaptic transmission in the adult mammalian CNS.

NgR1 is a regulator of activity-dependent synaptic strength. In the central nervous system, excitatory neurotransmission most commonly occurs at dendritic spines. Spines are highly motile structures and it is believed that their morphological plasticity reflects adaptive alterations in synaptic strength as a result of altered neural activity. Long term potentiation (LTP), a form of synaptic plasticity important for memory, leads to rapid spine actin polymerization. NgR1 functions as an inhibitory receptor for FGF-signaling (e.g., by inhibiting FGF-enhancement of long term potentiation in hippocampal neurons). Furthermore, NgR1 agonists (e.g., OMgp) modulate synaptic transmission via NgR1. Thus, NgR1 participates in activity-dependent regulation of synaptic strength and is an important regulator of structural neuronal plasticity in subjects, including adult subjects.

The compositions and methods herein are designed to overcome both effects of NgR1 so as to maximize neurite outgrowth, neuronal regeneration, activity-dependent synaptic strength, neuronal plasticity, and memory. Also provided are compositions and methods that promote the effects of NgR1 to downregulate activity-dependent synaptic strength.

Agents and Compositions that Modulate NgR1

Provided herein are agents that modulate NgR1 expression or NgR1 ligand binding. Such agents include small molecules, polypeptides, siRNAs, etc. By modulate is meant promoting, increasing or enhancing on the one hand or a inhibiting, decreasing, or reducing on the other hand. Such relative terms refer to a comparison to a control (e.g., in the absence of the agent).

By way of example, a polypeptide comprising a fragment of NgR1 is provided, wherein the NgR1 fragment is mutated or modified to have reduced FGF2 binding as compared to wild-type NgR1. Such amino acid mutations or modifications include, for example, substitutions or deletions of one or more amino acid residues within the FGF2 binding domain or a substitution or deletion of the complete FGF2 binding domain. The MAG and FGF2 binding sites on NgR1 are distinct. Thus, the NgR1 fragment can comprise the MAG binding site but lack the FGF2 binding site or may contain the MAG binding site with a modified FGF2 binding site. The modified or mutated NgR1 fragment comprises from about 100 to about 377 amino acid residues or any amount in between, including for example, about 310 to about 377. By reduced binding is meant at least about 10% lower than the amount or affinity of binding to wild-type NgR1. Reduced binding can include the complete elimination of FGF2 binding by the fragment or any amount between 10% and complete elimination. The Kd of wild-type NgR1-FGF2 interaction is in the range of 5-10 nM. Thus a reduction in binding can be characterized by a reduction of at least 10% in the Kd such that the NgR1 fragment or domain has a binding affinity for FGF2 in the range of 0.5 to 1 nM or less.

By way of example, the sequence for full-length rat NgR1 is as follows: mkrassggsr llawvlwlqa wrvatpcpga cvcynepkvt tscpqqglqa vptgipassq riflhgnris yvpaasfqsc rnltilwlhs nalagidaaa ftgltlleql dlsdnaqlrv vdpttfrglg hlhtlhldrc glqelgpglf rglaalqyly lqdnnlqalp dntfrdlgnl thlflhgnri psvpehafrg lhsldrlllh qnhvarvhph afrdlgrlmt lylfannlsm lpaevlvplr slqylrlndn pwvcdcrarp lwawlqkfrg sssevpcnlp qrlagrdlkr laasdlegca vasgpfrpfq tnqltdeell glpkccqpda adkasvlepg rpasagnalk grvppgdtpp gngsgprhin dspfgtlpgs aeppltalrp ggseppglpt tgprrrpgcs rknrtrshcr lgqagsgssg tgdaegsgal palacslapl glalylwtyl gpc (SEQ ID NO: 1). NgR1 sequences for other species including human are known and are very similar. Sequences for human, mouse, zebrafish, and chicken NgR1 are provided as SEQ ID NOs: 11-14. When specific examples are provided herein for rat NgR1 fragments, comparable sequences are considered to be disclosed for other species as well, as one of skill in the art could readily align the sequences and identify the comparable fragment.

The NgR1 fragment optionally comprises an amino acid sequence having at least about 80-99% identity to SEQ ID NO:2 (amino acid residues 27-311 of wild-type NgR1), SEQ ID NO:3 (amino acid residues 352-448 of wild-type NgR1), SEQ ID NO:8 (amino acid residues 378-473 of wild-type NgR1), SEQ ID NO:9 (amino acid residues 349-473 of wild-type NgR1), or SEQ ID NO:10 (amino acid residues 314-473 of wild-type NgR1).

More specifically, provided herein is a polypeptide comprising one or more fragments of NgR1, wherein the modified NgR1 fragment comprises an amino acid sequence having at least about 80%, 85%, 90%, 95%, or 99% identity to SEQ ID NO:2 or an amino acid sequence having at least about 80%, 85%, 90%, 95%, or 99% identity to SEQ ID NO: 3 or both. Also provided are modified NgR1 fragments comprising an amino acid sequence having at least about 80%, 85%, 90%, 95% or 99% identity to SEQ ID NOs:8, 9 or 10. The fragments are selected and optionally modified so as to provide reduced FGF2 binding as compared to wild-type NgR1. Alternatively, the polypeptide can comprise an NgR1 fragment comprising the amino acid sequence SEQ ID NO: 2 with one to about forty amino acid mutations or modifications, more particularly, about 20 amino acid mutations or modifications and, even more particularly, about 10 amino acid mutations or modifications. The polypeptide can comprise an NgR1 fragment comprising the amino acid sequence SEQ ID NO: 3 with one to about 40 amino acid mutations or modifications, more particularly, about 20 amino acid mutations or modifications and, even more particularly, about 10 amino acid mutations or modifications. The NgR1 fragment can comprise SEQ ID NO:2 and SEQ ID NO:3 with amino acid mutations or modifications.

Also provided is a chimeric polypeptide comprising the NgR1 fragment (e.g., the amino acid sequence of SEQ ID NO:2, 3, or both with one or more amino acid mutations or modifications that reduce FGF2 binding) and a NgR2 domain, wherein the chimeric polypeptide comprises a ligand binding domain. The chimeric polypeptide comprises myelin inhibitor binding properties of wild-type NgR1 and NgR2.

The full length sequence for rat NgR2 is provided as SEQ ID NO:4. NgR2 is a high affinity receptor selective for MAG. NgR2 binds MAG directly and with high affinity, Kd 1-2 nM. Soluble NgR2 has MAG antagonistic capacity and promotes neuronal growth on MAG and CNS myelin substrate in vitro (Venkatesh et al., J. Neurosci. 25:808-22, 2005). NgR1 binds directly to Nogo66 (Kd 7 nM) (Fournier et al., J. Neurosci. 22:8876-8883, 2001), OMgp (Kd 5 nM) (Wang et al., Nature 417:941-4, 2002), and MAG (Kd 8-20 nM) (Domeniconi et al., Neuron 35:283-90. 2002; Liu et al., Science 297:1190-3, 2002). The ligand-binding sites for Nogo66 and OMgp on NgR1 cannot be dissociated. The MAG binding site on NgR2 includes 13 amino acids located juxtaposed to the NgR2LRRCT domain. The NgR2 domain of the chimeric polypeptide comprises a full length NgR2 or a fragment or variant thereof. Optionally the NgR2 domain includes the MAG binding motif. Optionally, the NgR2 domain comprises the amino acid sequence of SEQ ID NO: 5 (amino acid residues 315-325 of full length NgR2) or the amino acid sequence of SEQ ID NO:6 (amino acid residue 315-327 of full length NgR2). Variants of these NgR2 fragments can also be used, including those having at least about 80%, 85%, 90%, 95%, or 99% identity to SEQ ID NO: 5 or 6 with one to four amino acid mutations, more particularly, about 3 amino acid mutations and, even more particularly, about 2 amino acid mutations.

For example, the chimeric polypeptide comprises a NgR2 domain comprising a 13-amino acid NgR2-peptide (Pro315-Ser327) juxtaposed to a first NgR1 ligand binding domain (e.g., the amino acid sequence of SEQ ID NO:2 with amino acid mutations or modifications that reduce FGF2 binding) is sufficient to provide high affinity MAG binding, and Nogo66 and OMgp binding capacity.

The ligand binding domain of the chimeric polypeptide comprises several leucine rich repeats (LRRs) and more specifically an N terminal LRR capping domain (called the LRRNT), a C terminal LRR capping domain (called the LRRCT), and at least one LRR between the LRRNT and the LRRCT. Thus the binding domain of the chimeric polypeptide can comprise LRRNT+LRR₁₋₆+LRRCT; however, additional LRRs can be present. The leucine rich repeats of the binding domain are not necessarily juxtaposed and can include intervening amino acid residues. The ligand binding domain binds a myelin-derived-growth-inhibitory protein. Specifically disclosed are chimeric proteins comprising a ligand binding domain of NgR1 or having at least about 85% (including, for example, 90, 95, 99%) sequence identity to the native NgR1 ligand binding domain. Alternatively, the ligand binding domain of the chimeric polypeptide comprises the ligand binding domain of NgR2, NgR3, or a sequence having at least about 85% (including, for example, 90, 95, 99%) sequence identity to the native NgR2 or NgR3 ligand binding domain.

Optionally, the chimeric polypeptide comprises the LRR cluster of NgR1 or a modification thereof and the 13 amino acids of NgR2 comprising the MAG binding site or a modification thereof. The chimeric polypeptide can comprise these two domains, wherein the NgR2 fragment is juxtaposed to the C terminus of the LRR cluster if NgR1. The chimeric polypeptide can further comprise, optionally at the C terminal end of the NgR2 domain, additional NgR1 residues (e.g., residues 352-448 (SEQ ID NO:3) from NgR1 or a modification thereof). This construct is referred to herein as NgR^{syn}. NgR^{syn} combines the high affinity ligand binding properties of NgR1 and NgR2. Moreover, NgR^{syn} shows a 2.7- and 2.5-fold greater binding of Nogo66 and OMgp compared to wild-type NgR1, a 6-fold enhanced binding of MAG compared to wild-type NgR1, and a 1.3-fold enhanced binding of MAG compared to wild-type NgR2. Taken together, NgR^{syn} binds with higher affinity to multiple inhibitors than wild-type NgR1 and NgR2 combined. The NgR1 domain of NgR^{syn} further comprises a mutation or modification to reduce FGF2 binding affinity. Thus, the chimeric polypeptide binds with higher affinity to multiple inhibitors than wild-type NgR1 and NgR2 combined, but also has reduced binding to FGF2 as compared to wild type NgR1. Thus, a chimeric polypeptide is provided comprising NgR1 residues C27-V311/NgR2 residues P315-N325/NgR1 residues P352-G448], wherein the NgR1 domain comprises a modification(s) that reduce FGF2 binding of the chimeric polypeptide or NgR1 domain as compared to FGF2 binding of wild-type NgR1. The fragments and chimeric polypeptides can be generated recombinantly using methods known in the art. Alternatively the fragments and chimeric polypeptides can be synthesized using protein synthesis techniques known in the art. Fragments can be fused together to form the chimeric polypeptides using polypeptide linkage techniques. Such techniques are also known in the art.

The disclosed chimeric polypeptides and the fragments thereof can be membrane bound or, in the alternative, can be soluble. Furthermore, the fragments and the polypeptides can be isolated and present relatively free of other dissimilar fragments and polypeptides.

The fragments and chimeric polypeptides include modifications designed to reduce FGF2 binding. However, other amino acid modifications and mutations can be silent mutations with limited or no effect on the function of the amino acid sequence. Amino acid sequence mutations and modifications typically fall into one or more of three classes: substitutional, insertional or deletional variants. Insertions include amino and/or carboxyl terminal fusions as well as intrasequence insertions of single or multiple amino acid residues.

Insertions ordinarily will be smaller insertions than those of amino or carboxyl terminal fusions, for example, on the order of one to four residues. Fusion derivatives can be made by fusing a first and second polypeptide sequence by cross-linking or by recombinant cell cultures transformed with DNA encoding the fusion polypeptide. Deletions are characterized by the removal of one or more amino acid residues from the polypeptide sequence and ordinarily are prepared by site specific mutagenesis of nucleotides in the DNA encoding the fragment, thereby producing DNA encoding the modified fragment, and thereafter expressing the DNA in recombinant cell culture. Substitutional variants are those in which at least one residue has been removed and a different residue inserted in its place. Techniques for making substitution mutations at predetermined sites in DNA having a known sequence are well known and include, for example, M13 primer mutagenesis and PCR mutagenesis. Amino acid substitutions are typically of single residues but can occur at a number of different locations at once; insertions usually will be on the order of about from 1 to 10 amino acid residues; and deletions will range about from 1 to 30 residues. Deletions or insertions can be made in adjacent pairs, i.e. a deletion of 2 residues or insertion of 2 residues. Substitutions, deletions, insertions or any combination thereof may be combined to arrive at a final construct.

Substantial changes in FGF2 binding are made by selecting modifications (such as non-conservative substitutions) that change (a) the structure of the polypeptide backbone in the area of the substitution, for example as a sheet or helical conformation, (b) the charge or hydrophobicity of the molecule at the target site or (c) the bulk of the side chain. For example, substitutions that in general are expected to produce the greatest changes in the protein properties will be those in which (a) a hydrophilic residue is substituted for (or by) a hydrophobic residue; (b) a cysteine or proline is substituted for (or by) any other residue; (c) a residue having an electropositive side chain is substituted for (or by) an electronegative residue; or (d) a residue having a bulky side chain is substituted for (or by) one not having a side chain in this case, (e) by increasing the number of sites for sulfation and/or glycosylation.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, is accomplished, for example, by deleting one of the basic residues or substituting one by glutaminyl or histidyl residues.

Amino acid modifications further include post-translational derivatizations, including for example deamination. Other post-translational modifications include hydroxylation, phosphorylation, methylation, acetylation, amidation.

Also provided are nucleic acids and vectors that encode the fragments or the chimeric polypeptides disclosed herein and nucleotide sequences complementary to the nucleic acids that encode the fragments or chimeric polypeptides. The expression vectors include the selected nucleic acid, wherein the nucleic acid is operably linked to an expression control sequence. One nucleic acid may encode one or more fragments or polypeptides (including for example, a fragment of NgR1 and/or a fragment of NgR2). For example, provided herein are nucleic acids and vectors comprising the nucleic acids, wherein the nucleic acid encodes NgR^{syn}, wherein the NgR^{syn} has reduced FGF2 binding as compared to wild-type NgR1. Cells, including cultured or isolated cells, comprising one or more vectors, wherein each vector encodes one or more fragments or chimeric polypeptides are also provided.

Also provided are isolated nucleic acids comprising a sequence that hybridizes under highly stringent conditions to all or any portion of a hybridization probe having a nucleotide sequence that comprises a nucleotide sequence that encodes a polypeptide or fragment disclosed herein or a complement of the encoding nucleotide sequence. The hybridizing portion of the hybridizing nucleic acid is typically at least 15 (e.g., 15, 20, 25, 30, 40, or more) nucleotides in length. The hybridizing portion is at least 80% (e.g., 90% or 95%) identical to the portion of the sequence to which it hybridizes. Hybridizing nucleic acids are useful, for example, as cloning probes, primers (e.g., PCR primer), or a diagnostic probe. Nucleic acid duplex or hybrid stability is expressed as the melting temperature or Tm, which is the temperature at which a probe dissociates from a target DNA. This melting temperature is used to define the required stringency conditions. If sequences are identified that are related and substantially identical to the probe, rather than identical, then it is useful to first establish the lowest temperature at which only homologous hybridization occurs with a particular concentration of salt (e.g., SSC or SSPE). Assuming that a 1% mismatching results in a 1° C. decrease in Tm, the temperature of the final wash in the hybridization reaction is reduced accordingly (for example, if sequences having more than 95% identity are sought, the final wash temperature is decreased by 5° C.). In practice, the change in Tm can be between 0.5 and 1.5° C. per 1% mismatch. Highly stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. Salt concentrations and temperatures can be varied to achieve the optimal level of identity between the probe and the target nucleic acid. Additional guidance regarding such conditions is readily available in the art, for example, in Molecular Cloning: A Laboratory Manual, Third Edition by Sambrook et al., Cold Spring Harbor Press, 2001.

Also disclosed are vectors comprising the nucleic acids described herein. Thus, provided is a vector that comprises a nucleic acid that encodes one or more of the fragments or chimeric polypeptides described herein. Optionally, the nucleic acid of the vector is operably linked to an expression control sequence (e.g., a promoter or enhancer or both). Suitable expression vectors are well known to those of skill in the art and commercially available from a variety of sources such as Novagen, Inc., Madison, Wis.; Invitrogen Corporation, Carlsbad, Calif.; and Promega Corporation, Madison, Wis.

A cultured cell comprising the vector is also provided. The cultured cell can be a cultured cell transfected with the vector or a progeny of the cell, wherein the cell expresses one or more fragments or chimeric polypeptides described herein. Suitable cell lines are known to those of skill in the art and are commercially available, for example, through the American Type Culture Collection (ATCC).

The transfected cells can be used in a method of producing one or more fragments or chimeric polypeptides. The method comprises culturing a cell comprising the vector under conditions that allow expression of the fragment or polypeptide, optionally under the control of an expression sequence. The fragment or polypeptide can be isolated from the cell or the culture medium using standard protein purification methods.

The nucleic acids and fragments and polypeptides are described herein relative to sequence similarity or identity as compared to the naturally occurring NgR1, NgR2, and the like. Those of skill in the art readily understand how to determine the sequence similarity and identity of two polypeptides or nucleic acids. For example, the sequence similarity can be calculated after aligning the two sequences so that the identity is at its highest level. Alignments are dependent to some extent upon the use of the specific algorithm in alignment programs. This could include, for example, the algorithm of Smith and Waterman Adv. Appl. Math. 2: 482 (1981), the alignment algorithm of Needleman and Wunsch, J. Mol. Biol. 48: 443 (1970), by the search for similarity method of Pearson and Lipman, PNAS USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), BLAST and BLAST 2.0 and algorithms described by Altschul et al., Nucleic Acids Res. 25:3389-3402, 1977; Altschul, et al., J. Mol. Biol. 215:403-410, 1990; Zuker, M. Science 244:48-52, 1989; Jaeger et al. PNAS USA 86:7706-7710, 1989 and Jaeger et al. Methods Enzymol. 183:281-306, 1989. Each of these references is incorporated by reference at least for the material related to alignment and calculation of identity or similarity. It is understood that any of the methods typically can be used and that in certain instances the results of these various methods may differ. Where sequence similarity is provided as, for example, 95%, then such similarity must be detectable with at least one of the accepted methods of calculation.

Compositions comprising one or more of the fragments or any combination of the fragments are provided. For example, such a composition comprises a fragment or fragments of NgR1 or a variant(s) thereof and a fragment or fragments of NgR2 or a variant(s) thereof. The compositions can further comprise a pharmaceutically acceptable carrier or a culture medium.

As described above, the compositions can also be administered in vitro or in vivo in a pharmaceutically acceptable carrier. By pharmaceutically acceptable is meant a material that is not biologically or otherwise undesirable, i.e., the material may be administered to a subject, along with the polypeptide, small molecule, or nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. Suitable carriers and their formulations are described in Remington: The Science and Practice of Pharmacy, 21st Edition, David B. Troy, ed., Lippicott Williams & Wilkins (2005). These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Optionally, the compositions provided herein further comprise FGF2. The concentration of FGF2 in the composition can be selected from about 10 nanograms/ml to about 200 nanograms/ml, and more specifically from about 50 to 100 nanograms/ml.

Also provided herein are agents (including polypeptides) that activate NgR1 expression or activity. Agents that activate NgR1 expression or activity include NgR1 agonists. As used herein, the term NgR1 agonist refers to NgR1 agonists and analogues and derivatives thereof, including, for example, natural or synthetic functional variants which have NgR1 biological activity, as well as fragments of an NgR1 agonist having NgR1 biological activity. For example, one way to regulate NgR1 cell surface expression is via metalloproteinase inhibitors. NgR1 is shed from the cell surface by metalloproteinases, and their inhibition increases NgR1 cell surface expression.

The fragments, compositions, nucleic acids, vectors, and expression systems taught herein can be used to modulate neurite outgrowth, neuronal regeneration, activity-dependent synaptic strength, neorual plasticity, and memory in vitro or in vivo. Neurite outgrowth is the process of developing new neurons or extending existing neurons. As an example of a method of promoting neurite outgrowth, provided herein is a method comprising contacting a neuron or a population of neurons with a fragment of NgR1, wherein the NgR1 fragment is mutated or modified to have reduced FGF2 binding as compared to wild-type NgR1. The method can further comprise contacting the neuron or population of neurons with an NgR2 fragment disclosed herein and with FGF2. Contacting the neuron with the NgR1 fragment can occur prior to, at the same time, or after contacting with the NgR2 fragment and the FGF2. Contacting with the NgR2 fragment can occur prior to, at the same time or after contacting with the FGF2 or with a chimeric polypeptide or composition comprising the NgR1 fragment.

To promote neurite outgrowth, neuronal regeneration, synaptic strength, neuronal plasticity, or memory, in vivo or in vitro, the neurons or population of neurons can be contacted with an agent that promotes expression or activity of NgR1. For example, a chimeric polypeptide disclosed herein can be used, optionally with further contacting the neurons with FGF2. Alternatively, the neurons or population of neurons can be contacted with a composition comprising any of the NgR fragments, chimeric polypeptides, and, optionally FGF2 as disclosed herein.

Also provided herein is a method of treating a central nervous system disease or a injury in a subject comprising administering to the subject a fragment or fragments, a chimeric polypeptide or polypeptides, or a composition or compositions taught herein, optionally, in combination with FGF2. For example, a method of treating a subject with a neurodegenerative or a central nervous system disease or condition includes the steps of administering to the subject an agent that blocks NgR1 expression or NgR1 ligand binding and administering to the subject FGF2 or an agonist of FGF2. Such methods promote regeneration, neuronal plasticity, synaptic strength and the like in the subject or ameliorate one or more symptoms of the disease or condition. Symptoms to be ameliorated include, for example, a reduction in tremors, a reduction in paralysis, an increase in coordination or dexterity, as increase in strength, and the like. CNS disorders include diseases and injuries such as, for example, stroke, brain and spinal cord injury, hypoxic events, subdural hematoma, multiple sclerosis, amyotrophic lateral sclerosis, Parkinson's disease, Alzheimer's disease or other dementias, or other neurodegenerative disorders or central nervous system injuries. Such a method would also be useful to address the effects on aging on a subject's brain, in the absence of a specific disease or injury.

Provided herein is a method of promoting activity-dependent synaptic strength, optionally without affecting basal levels of synaptic strength. The method, which can be performed in vivo or in vitro, includes contacting a neuron (e.g., a postsynaptic neuron) with an agent that blocks NgR1 expression or NgR1 ligand binding. The agent can be one that blocks a ligand binding, such as oligodendrocyte myelin glycoprotein (OMgp) binding, to NgR1. Such agents that block ligand/NgR1 binding include small molecules, polypeptides (e.g., the polypeptides taught herein), fragments or variants of NgR1 (e.g., soluble fragments or variants lacking the GPI region), wherein the fragment or variant competitively binds OMgp (or other ligand) or otherwise interrupts binding of ligand to NgR1, perhaps through steric hinderance with the OMgp binding site on NgR1.

Reduction or inhibition of NgR1 can comprise inhibiting or reducing expression of NgR1 mRNA or NgR1 protein, such as by administering antisense molecules, triple helix molecules, ribozymes and/or siRNA. NgR1 gene expression can also be reduced by inactivating the NgR1 gene or its promoter. The nucleic acids, ribozymes, siRNAs and triple helix molecules for use in the provided methods may be prepared by any method known in the art for synthesis of DNA and RNA molecules. These include techniques for chemically synthesizing oligodeoxyribonucleotides and oligoribonucleotides well known in the art such as for example solid phase phosphoramide chemical synthesis. Alternatively, RNA molecules may be generated by in vitro and in vivo transcription of DNA sequences encoding the nucleic acid molecule. Such DNA sequences may be incorporated into a wide variety of vectors, which incorporate suitable RNA polymerase promoters. Antisense cDNA constructs that synthesize antisense RNA constitutively or inducibly, depending on the promoter used, can be introduced stably into cell lines.

NgR1 antagonists also include antibodies, soluble domains of NgR1 and polypeptides that interact with NgR1 to prevent NgR1 activity. Thus, inhibitors of NgR1 include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more. Inhibitory peptides include dominant negative mutants of a NgR1. Dominant negative mutations (also called antimorphic mutations) have an altered phenotype that acts antagonistically to the wild-type or normal protein. Thus, dominant negative mutants of NgR1 act to inhibit the normal NgR1 protein. Such mutants can be generated, for example, by site directed mutagenesis or random mutagenesis. Proteins with a dominant negative phenotype can be screened for using methods known to those of skill in the art, for example, by phage display.

Nucleic acids that encode the aforementioned peptide sequences are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants. Such peptides or proteins are selected based on their ability to reduce or inhibit expression or activity of NgR1.

Proteins that inhibit NgR1 also include antibodies with antagonistic or inhibitory properties. In addition to intact immunoglobulin molecules, fragments, chimeras, or polymers of immunoglobulin molecules are also useful in the methods taught herein, as long as they are chosen for their ability to inhibit NgR1. The antibodies can be tested for their desired activity using in vitro assays, or by analogous methods, after which their in vivo therapeutic or prophylactic activities are tested according to known clinical testing methods.

Also provided herein are functional nucleic acids that inhibit expression of NgR1. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of NgR1.

Functional nucleic acids are nucleic acid molecules that have a specific function, such as binding a target molecule or catalyzing a specific reaction. Functional nucleic acid molecules can interact with any macromolecule, such as DNA, RNA or carbohydrate chains. Thus, functional nucleic acids can interact with mRNA or genomic DNA. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules can be found in U.S. Pat. Nos. 5,168,053; 5,624,824; 5,683,873; 5,728,521; 5,869,248; and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

The capacity of an agent to promote activity-dependent synaptic strength can be demonstrated by one of skill in the art using any number of available methods including, for example, using electrophysiological techniques or morphological analysis of dendritic structure (see Examples). By activity-dependent is meant that the change in synaptic strength is dependent on excitatory input to the cell. Such excitatory input can be achieved by applying an excitatory stimulus (e.g., an electrical impulse or a ligand that stimulates excitatory postsynaptic potentials). One option is to contact the neuron with FGF-2.

A method of promoting memory (e.g., long term memory) in a subject is also provided herein. The method includes administering to the subject an agent that blocks NgR1 expression or NgR1 ligand binding and, optionally, also includes administering to the subject a neuroexcitatory input (e.g., FGF2 or an agonist of FGF2). Memory can be assessed using electrophysiological measures (e.g., evaluation of long-term potentiation) or behavioral measures (i.e., tests for memory and learning).

Additional method provided herein relate to reducing activity-dependent synaptic strength. Such a method includes contacting a postsynaptic neuron with an agent that promotes NgR1 expression or NgR1 ligand binding. Relatedly, a method of treating a seizure disorder in a subject is provided that includes administering to a subject an agent that promotes NgR1 expression or NgR1 ligand binding, and, optionally, further including administering to the subject a second anti-seizure medication. An effect on seizure activity can be assessed by one of skill in the art based on EEG data, number of seizures, etc.

Screening methods are also provided herein to identify or characterize agents for use in these methods. For example, provided is a method of screening for a molecule that modulates activity-dependent synaptic strength, which includes the steps of contacting a NgR1-containing cell with the agent to be tested, providing an agent that stimulates activity (e.g., a small molecule, a polypeptide, a neurotransmitter, or an electrical stimulus), and detecting a change in activity in the presence of the agent to be tested. An increase in activity-dependent synaptic strength indicates an agent that increases synaptic strength, whereas a decrease in synaptic strength indicates an agent that increases synaptic strength.

The exact amount of the compositions or agents used in the methods herein will vary from subject to subject, depending on the species, age, weight and general condition of the subject, the severity of the disease or condition being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

One of skill in the art selects the amount and frequency of contact or administration with the agent or composition (e.g., NgR1 fragment, the NgR2 fragment, the chimeric polypeptide, the FGF, or any combination thereof) that optimizes the desired outcome (e.g., promotion of neurite outgrowth, increase in activity dependent synaptic strength, promoting memory, or ameliorating a particular symptom or set of symptoms injury or disease). The concentration of FGF2 is selected from about 10 nanograms/ml to about 200 nanograms/ml, and more specifically from about 50 to 100 nanograms/ml.

The compositions may be administered orally, parenterally (e.g., intravenously), intraventricularly, intrathecally (for example into the lumbar cistern), direct injection into the central nervous system (e.g., by stereotaxic administration or image-guided administration), by intramuscular injection, by intraperitoneal injection, transdermally, topically or the like. Upon injury or insult to the central nervous system, the compositions could be administered locally, for example, when a surgical procedure in required to stabilize the spine, relieve intracerebral pressure, and the like.

Injectables can be prepared in conventional forms, either as liquid solutions or suspensions, solid forms suitable for solution of suspension in liquid prior to injection, or as emulsions. Administration can involve the use of a slow release or sustained release system such that a constant dosage is maintained. Thus, the materials may be in solution, in suspension, or may be incorporated into microparticles, liposomes, or cells. These may be targeted to a particular cell type via antibodies, receptors, or receptor ligands.

The fragments or polypeptides provided herein can be administered by transfection of a cell with a nucleic acid or a vector encoding the fragment(s) or polypeptide(s) so that the cell expresses the nucleic acid(s) and thereby provides the fragment(s) or polypeptide(s) indirectly to the neuron or subject. Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome. Other general techniques for integration into the host genome include, for example, systems designed to promote homologous recombination with the host genome.

Methods of administering the nucleic acids of the invention are also provided. There are a number of compositions and methods which can be used to deliver nucleic acids to cells, either in vitro or in vivo using systems such as a viral based delivery systems or a non-viral based delivery system. For example, the nucleic acids can be delivered in a saline solution or through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are known in the art.

Agents that promote crossing the blood brain barrier can also be used to facilitate entry of the agents or compositions into the central nervous system. Thus, when the agents or compositions are administered peripherally, a blood brain barrier permeabilizer can be used. Blood brain barrier permeabilizers are known in the art and include, by way of example, bradykinin and the bradykinin agonists described in U.S. Pat. Nos. 5,686,416; 5,506,206 and 5,268,164 (such as NH2-arginine-proline-hydroxyproxyproline-glycine-thienylalanine-serine-proline-4-Me-tyrosine.ψ(—CH2NH)-arginine-COOH). The agent can optionally be conjugated to a transferrin receptor antibody as described in U.S. Pat. No. 6,329,508; 6,015,555; 5,833,988 or 5,527,527 to promote into the central nervous system.

For each method of treatment provided herein, a step of selecting a subject in need of such effect is contemplated and disclosed. Also contemplated and disclosed is a step of monitoring the outcome of the treatment in the subject. Thus, for example, provided herein is a method of promoting memory in a subject comprising administering to the subject an agent that blocks NgR1 expression or NgR1 ligand binding. Such method can further comprise selecting a subject in need of enhanced memory, can further comprise testing the memory of the subject before and/or after treatment, or can comprise both the selecting step and the testing step or steps.

Disclosed are methods of making the agents and compositions (including fragments and chimeric polypeptides taught herein) by the steps of culturing a cell comprising the vector of the invention under conditions for expressing the receptor protein and isolating the protein.

As used throughout, terms like blocking binding refer to a reduction in binding (as with competitive binding) or a complete ablation (as with complete sequestering).

Incorporated herein is PCT/US2004/010328, filed Apr. 2, 2004, which published as WO/2004/090103. This international application is incorporated in its entirety for the methods, compositions, and sequences taught therein. The fragments, polypeptides, compositions, nucleic acids, vectors, and cells provided herein can be used in methods and with compositions (including, for example, proteoglycans like syndecan) as taught in the international application.

A number of embodiments have been described. Nevertheless, it will be understood that various modifications may be made. For example, a NgR1 fragment described herein can be administered concurrently or sequentially with FGF2 as well as other compounds or agents, such as sulfated proteoglycans or glycosaminoglycan (GAG) chains. Accordingly, other embodiments are within the scope of the following claims.

The following examples are intended as illustrative only since numerous modifications and variations therein will be apparent to those skilled in the art.

### EXAMPLES

### Example 1

### NgR1, in Conjunction with Specific Proteoglycans, Functions as a Negative Regulator of Neuronal FGF1 and FGF2 Signaling

Reagents

The following reagents were purchased from the sources indicated: OptiMEM, DMEM, Neurobasal medium, B27 supplement, fetal bovine serum (FBS), Pen/Strep, G418, and glutamine (Invitrogen, San Diego, Calif.); mouse NogoR/Fc, human FGFR1α(IIIb)/Fc, TROY/Fc, EphrinB1/Fc, FGF1 (R & D Systems); FGF2 (Peprotech, Rocky Hill, N.J.); class III β-tubulin antibody (TuJ1; Promega, Madison, Wis.); Alexa red anti-mouse IgG and Alexa green anti-rabbit IgG (Molecular Probes, Eugene, Oreg.); anti-AP (American Research Products, Belmont, Mass.); Lipofectamine 2000, mammalian tissue protease inhibitor mixture, insulin (Sigma, St. Louis, Mo.); Protein G Plus/Protein A-agarose beads (Oncogene, San Diego, Calif.); HRP/ECL detection system (Amersham Biosciences, Piscataway, N.J.).

Tissue Culture

Rat pheochromocytoma PC12 cells were maintained in DMEM supplemented with 10% FBS and 5% horse serum. Stable clones were prepared by transfecting PC12 cells with the pcDNA3 containing rat NgR1 cDNA clone followed by selection using the drug G418.

PC12 Cell Neurite Outgrowth

Cells were plated at low density (10,000 cells/well of 24 well plate) on poly-lysine (0.1 mg/ml in PBS) coated plate. After overnight culture, cells were cultured in low serum media (DMEM supplemented with 0.5% FBS and 0.25% horse serum). After 24 hr growth factors were added to the cells in the low serum medium for 4 days. Fresh growth factors were added the second day with the medium change.

PC12 cells stably expressing full-length NgR1 do not differentiate in the presence of FGF. PC12 cells were differentiated in the presence of FGF2 (25 ng/ml or 50 ng/ml) or NGF for 4 days and then stained with TUJ1 and anti-NgR1. Wild-type (WT) PC12 cells show robust neurite outgrowth in the presence of FGF2 (25 ng/ml and 50 ng/ml) and NGF (25 ng/ml). A PC12 cell line stably expressing NgR1 (PC12-NgR1-17) shows a greatly reduced response to FGF2. The response to NGF is reduced but to a lesser extent than for FGF2. Thus, NgR1 overexpressing PC12 cells do not differentiate and form neurite like processes, in marked contrast to NgR1 negative PC12 cells which form neurites. This shows that NgR1 negatively regulates FGF2 signaling in PC12 cells.

FGF2-NgR1-Fc Cross-Linking

¹²⁵I-FGF2 (specific activity: approximately 50 μCi/ug) was purchased from MP Biochemicals. NgR1-Fc (13 nM) was incubated with 40 nM ¹²⁵I-FGF2 with or without the indicated concentrations of cold FGF2 or insulin for 2 h at room temperature in PBS in a volume of 15 μl. FGFR1α(IIIb)/Fc was used as a positive control. Cross-linking was initiated by adding BS³ (bis[sulfosuccinimidyl] suberate) to a final concentration of 2 mM and incubating for 30 min at room temperature. Reactions were stopped by adding 2 μl of 1 M Tris-HCl (pH 7.4) and analyzed by SDS-PAGE. Gels were subsequently fixed, dried, and subjected to autoradiography at −70° C. using Kodak BioMax MS film.

Transfection of PC12 Cells and Western Blot Analysis

For analysis of activation of MAPK, MEK, and Akt, cells were plated on poly-lysine coated 24-well plate at 50,000 cells/well. After overnight culture cells were serum-starved in DMEM with 0.1% FBS for 24 hr. Cells were treated with growth factors for 5 min at RT, lysed in 2× Laemmli sample buffer and boiled for 10 min before separation using a 10% SDS-PAGE gel. Cell lysates were subjected to Western blotting using a set of anti-p44/42 ERK and anti-phospho-specific p44/42 ERK antibodies, a set of anti-MEK and anti-phospho-specific MEK antibodies, and a set of anti-Akt and anti-phospho-specific Akt antibodies (Cell Signaling Technology, Danvers, Mass.), respectively. Filters were stripped and reprobed with anti-actin antibody.

For expression of FGF2 signaling molecules in PC12 cells and cells stably expressing NgR1 (clone PC12-NgR1-17), cells were transfected using the Amaxa Biosystems (Köln, Germany) nucleofection technology. Briefly, 2×10⁶ cells in 100 μl of Cell Line Nucleofector solution were mixed with 2 μg of plasmid DNA (GFP, constitutively active H-ras (ca-ras), FGF-receptor 1 (FGFR1) or FGFR3) and transferred into a cuvette for electroporation. Cells were transfected using the U-29 pulsing parameter. Transfected cells were plated on 24-well plate coated with poly-lysine and after 2 days assayed for FGF2 responsiveness by anti-phosphoErk1/2 immunoblotting as described above.

Cortical Neuronal Culture Preparation

Cultures were prepared from cortical tissue obtained from embryonic day 18 rat fetuses. Cortical pieces were dissociated with 0.05% trypsin for 4 min at 37° C. in neurobasal medium, 0.5 mM EDTA, and 0.01% DNase I with gentle agitation, followed by gentle trituration in DMEM with 10% FBS. Pooled cell suspensions were centrifuged at 100×g for 5 min, resuspended in fresh medium. Trituration was repeated twice with fresh medium and counted on a hemocytometer. Cortical neurons were transfected using the Amaxa Biosystems nucleofection technology. Briefly, 4×10⁶ cells in 100 μl of Rat Neuron Nucleofector solution were mixed with 10 μg of pEGFP-N1 plasmid DNA or 10 μg of pMT21-NgR1 and transferred into a cuvette for electroporation. Cells were transfected using the 0-03 pulsing parameter, transferred into 0.5 ml of 37° C. prewarmed DMEM medium containing 10% FBS. Cells (1.5×10⁵/well) were plated on 6-well plate coated with poly-lysine (0.5 mg/ml in 0.1 M borate buffer, pH 8.5) and subsequently with laminin (20 μg/ml) in Neurobasal medium. After overnight culture media was changed to low B27 medium (Neurobasal medium containing 1 ul/ml B27 supplement, 25 mM glucose, 1 mM glutamine, 50 U/ml penicillin/50 μg/ml streptomycin). Twenty four hours after transfection, the low B27 medium with or without FGF2 (15 ng/ml) was added to the cells and neuronal cells were cultured for 2 days.

Immunocytochemistry

The cultures were fixed in PBS containing 4% paraformaldehyde and 0.4 M sucrose in PBS for 30 min at RT. Cells were incubated with blocking solution (PBS containing 2.5% horse serum) for 30 min. Subsequently, cells transfected with the NgR1 plasmid were incubated with anti-NgR1 antiserum at the dilution of 1:1,000 for 2 hr at RT, followed by Alexa green anti-rabbit IgG (1:1,000) for 30 min in the blocking solution. Subsequently, neurons were immunostained with anti-βIII tubulin at the dilution of 1:1,000 in the blocking buffer containing 0.2% Triton X-100 overnight at 4° C., followed by Alexa red anti-mouse IgG (1:1,000) for 30 min in the blocking solution containing 0.2% Triton X-100.

Quantitative Analysis of Neurons

All fluorescence pictures were taken with an IX71 Olympus (Melville, N.Y.) inverted microscope attached at the side to a DP70 digital camera. For quantification of branches, pictures of dissociated cortical neurons with processes equal to or longer than approximately two cell body diameter were taken. The number of axon branches >20 μm in length were counted. Neurite length was measured from digitized images by UTHSCSA Image Tool for Windows, version 3.0.

Ligand Binding to NgR1 in COS Cells

Techniques for ligand binding to NgR1 on COS cells is described in detail in Venkatesh et al. (2005) Journal of Neuroscience 25:808-22, which is incorporated herein by reference in its entirety at least for the methods taught therein. Briefly, receptor constructs were expressed transiently in COS-7 cells in 24-well plates coated with poly-D-lysine (PDL; 50 μg/ml), using Lipofectamine 2000. At 24 h after transfection the cells were rinsed and incubated for 75 min at ambient temperature with alkaline phosphatase tagged FGF1, FGF2, FGF4, FGF8, FGF9, and FGF21. To monitor bound ligand, plates were developed with NBT/BCIP substrate; the color reaction was stopped by two rinses in PBS. For quantification of ligand binding the cells were processed as described above; after ligand incubation the cells were rinsed in HBHA and lysed in 20 mM Tris, pH 8.0, 0.1% Triton X-100. The lysates were incubated at 65° C. for 60 min and spun at 10,000×g for 5 min. The relative AP activity in supernatants was normalized to cell surface receptor expression, using anti-myc (1:1000), anti-NgR1 (1:1000), anti-NgR2 (1:1000), or anti-NgR3 (1:200) antibodies, as described previously (Giger et al., Neuron 21:1079-92, 1998). Scatchard plot analysis was performed analogous to a previous study (Kolodkin et al., Cell 90:753-62, 1997).

### Example 2

### Identification of FGF2 and NgR1 Interactions

Identification of Specific NgR1 Region Required for the FGF2 Interaction.

NgR1 heparin binding motif [TGPRRRPGCSRKNRTRL (SEQ ID NO:7), residues 413-427] in the C-terminal stalk region is necessary to attenuate FGF2 and NGF signaling in PC12 cells. NgR1 lacking the heparin binding motif (Delta pos. box) does not suppress FGF2 (25 ng/ml) or NGF (25 ng/ml)-mediated differentiation in PC12 cells. As a control wild-type PC12 cells were mixed with different cell lines, exposed to growth factor for 4 days and then immunolabelled with anti-NgR1 and TuJ1. Neurite outgrowth of NgR1 positive and negative PC12 cells was quantified as described above.

NgR1 is a Negative Regulator of FGF2-Induced MAP-Kinase Signaling.

In wild-type PC12 cells, FGF2 elicits a dose-dependent activation of the MAP-kinase pathway. A FGF2 dose-response curve at 0, 0.1, 0.5, 1.5, 5, and 50 ng/ml was performed. Activation of the MAP-kinase pathway was assessed by anti-pERK1/2 (p-MAPK) and anti-pMEK immunoblotting and normalized to total ERK1/2 (MAPK) or MEK. In marked contrast to wild-type PC12 cells, PC12 cells overexpressing NgR1 do not show activation of the MAP-kinase pathway in the presence of FGF2. Similar experiments with NGF and EGF show that NgR1 attenuates NGF but not EGF elicited MAP-kinase signaling at low doses 1.5 and 5 ng/ml compared to wild-type PC12 cells.

Similar to FGF2, acidic FGF (FGF1) signaling at 10 ng/ml and 40 ng/ml is significantly decreased in NgR1 overexpressing PC12 cell lines as assessed by anti-pERK1/2 immunoblotting.

To determine whether MAAPK signally could be restored, wild-type PC12 cells (WT-PC12) and PC12 cells stably expressing NgR1 (PC12-NgR1-17) were transfected with GFP (control), constitutively active H-ras (ca-ras), FGF-receptor 1 (FGFR1) or FGFR3 plasmid DNA and assayed for FGF2 responsiveness by anti-phosphoErk1/2 immunoblotting. Transfection of caRas, FGFR1, and FGFR3 restores the MAPK pathway in the NgR1 overexpressing cells.

Characterization of NgR-FGF Binding.

NgR1 supports high affinity binding of alkaline phosphatase tagged FGF1 and FGF2. The Kd for NgR1-FGF2 binding is about 10 nM, whereas the Kd for NgR1-FGF1 binding is about 5 nM. FGF4 shows weak binding to NgR1. FGF8b and FGF21 do not interact with NgR1. None of these FGF ligands interacts with NgR2. As a positive control, MAG-Fc binds to NgR1 and NgR2. FGF2 binding to NgR1 is not altered in the presence of heparin. Consistent with this, deletion of the heparin binding motif within NgR1 (Δ 29) does not alter binding to NgR1. Deletion of 41 amino acids from the C-terminus of FGF2 (Δ 41) results in a mutant that no longer binds to NgR1. The NgR1 leucine-rich repeat cluster (amino acids 1-310) is sufficient to support AP-FGF2 binding (i.e. deletion of the unique region does not alter FGF2 binding). The LRRCT domain and the LRR repeats 1-8 are necessary for FGF2 binding.

FGF2 binds directly and specifically to NgR1. Soluble NgR1-Fc, FGFR-Fc, TROY-Fc, or ephrinB1-Fc were incubated with [¹²⁵I]-FGF2 in the presence or absence of competitor (80- and 160-fold excess of cold (unlabeled) FGF2 or 80-fold excess of insulin. Ligand receptor complexes were cross-linked and analyzed by SDS-PAGE.

Ectopic Expression Of NgR1 in Primary Cortical Neurons Suppresses FGF2 Induced Axonal Branching.

E18 rat cortical neurons were transfected with NgR1 or GFP plasmid DNA and exposed to FGF2 (25 ng/ml) for 2 days. Cells were fixed and stained with anti-NgR1 or TuJ1. Cortical neurons overexpressing recombinant NgR1 do not show a branching response in the presence of FGF2.

### Example 3

### NgR1 Role in Regulation of Synaptic Transmission in Adult Hippocampus

AP-Fusion Protein Binding

Binding studies with AP-tagged ligands to transiently transfected COS cells or brain tissue sections were performed as follows. Human placental alkaline phosphatase (AP)-tagged fusion proteins were constructed by standard PCR cloning using the Tth-DNA polymerase. AP-sNgR1, AP-sNgR2, AP-sNgR3, AP-Fc, AP-Nogo66, and AP-NiG have been described previously (Venkatesh et al., J. Neurosci. 25:808-822, 2005). Additional constructs included AP-sNgR1^{NT-LRR-CT} (Ala²⁴-V³¹¹), AP-sNgR [symbol] (Ser³⁰-Thr³¹⁴), AP-sNgR3^{NT-LRR-CT} (Ser²²-Pro³⁰⁷), AP-sNgR1^{CT+stalk} (Phe²⁷⁸-Glu⁴⁴⁵), AP-SNgR2^{CT+stalk} (Ala²⁷⁹-Ser³⁹⁷), and AP-sNgR3^{CT+stalk} (Phe²⁷³-Val⁴¹³). Ligands used for Nogo receptor binding studies included AP-FGF2 (mouse), AP-FGF1 (human), AP-FGF4 (mouse), AP-FGF8 (mouse), AP-FGF9 (mouse), AP-FGF21 (mouse), AP [symbol] PTN/HB-GAM (Origene, Rockville, Md.), AP-VEGF165 (M. Klagsburn), and MAG-Fc (R&D Systems, Minneapolis, Minn.).

To monitor binding of soluble Nogo receptors to brain tissue sections in situ, embryonic (E18) and neonatal (P1-P7) rat or mouse brains were flash frozen in dry ice cooled isopentane, cryo-sectioned at 20 μm, fixed for 8 minutes in 100% methanol at −15° C., and rinsed in PBS. Soluble receptor fusion proteins were tagged N-terminally to human placental alkaline phosphatase (AP) and expressed in transiently transfected HEK293T cells. Tissue sections were incubated at room temperature for 75 minutes with conditioned cell culture supernatants containing 3-5 nM of AP-fusion proteins. In some experiments NgR1-Fc (2.5 μg, R&D Systems) was used and detected with anti-human Fc conjugated to AP (Chemicon, Temecula, Calif.). Sections were processed and developed as described (Giger et al., Neuron 25:29-41, 2000). Brain tissue sections of unfixed E18 or P3-P7 wild-type mice and p75^{-/-exon III} (Lee et al., Cell 69:737-49, 1992), p75^{-/-exonIV} (von Schack et al., Nat. Neurosci. 4:977-8, 2001), NgR1−/− (Zheng et al., Neuron 38:213-224, 2003), GalNAcT^{-/-} (Liu et al., J. Clin. Invest. 10:497-505, 1999) and GD3S^{-/-} mutants (Kawai et al., J. Biol. Chem. 276:6885-6888, 2001) were assayed for AP-sNgR^{CT+stalk}, AP-sNgR2^{CT-stalk}, and AP-sNgR3^{CT+stalk} binding as described above.

To directly assay binding of soluble receptor fusion proteins to previously identified components of the NgR1 receptor complex or NgR family members, COS-7 cells were transiently transfected with plasmid DNA encoding p75, TROY, Lingo-1 (Origene), L-MAG, OMgp, a chimeric form of human Nogo66 fused to the transmembrane and cytoplasmic portion of rat neuropilin-1 (Nogo66-Npn1), rat NgR1, rat NgR2, or rat NgR3. Cell surface expression of recombinant proteins was confirmed by immunocytochemistry or binding of MAG-Fc. Binding studies were carried out as described previously (Venkatesh et al., J. Neurosci. 25:808-822, 2005).

To assess whether the association of AP-sNgR1^{CT+stalk} or AP-sNgR3^{CT+stalk} to brain tissue sections is mediated by protein-protein interactions, prior to incubation with AP-fusion proteins, brain sections were preincubated at 75° C. for up to 3 hours or treated with trypsin (10 units/37° C. for 30 min), using the Neuropilin-2 ligand AP-Sema3F as a positive control (Kantor et al., Neuron 44:961-75, 2004). To explore whether glycoconjugates participate in binding of soluble Nogo receptors to brain tissue, sections were enzymatically treated with N-acetylglucosaminidase, Vibrio cholerae neuraminidase, heparinase III (Flavobacterium heparinium), chondroitinase ABC (all from Calbiochem, San Diego, Calif.), glycopeptidase F (New England Biolabs, Beverly, Mass.), or endoneuraminidase-N, prior to incubation with the AP-fusion proteins.

For quantification of ligand binding following glycosidase treatment, six consecutive brain tissue sections at the level of the hippocampus (20 μm) were lifted per microscope slide and either incubated with enzyme or with enzyme buffer only following the manufacturer's instructions. AP-fusion proteins were bound as described above and after 75 mins unbound ligand was removed by several rinses in PBS. Tissue sections were then scraped into a test tube with 200 μl HEPES buffered saline, pH 7.0. Endogenous phosphatases were heat inactivated at 65° C. for 2 hrs, and binding of fusion proteins was quantified by measuring AP enzymatic activity at OD405.

FGF2-NgR1-Fc Binding Studies

NgR1-Fc (0.5 μg, R&D Systems) and AP-tagged ligands (1.5 nM final conc.) were mixed in DMEM containing 0.1% BSA and protease inhibitor cocktail and were incubated at 4° C. for 2 h. AP-ligands bound to NgR1-Fc were precipitated with Protein A/G agarose beads after 2 h at 4° C. Samples were rinsed extensively in washing buffer (20 mM Tris-HCl, pH 7.5, 150 mM NaCl, 1% NP40, 5 mM EDTA) and analyzed by Western blotting using an anti-AP antibody. For cross-linking experiments, NgR1-Fc, FGFR1D(IIIb)/Fc, TROY-Fc, or ephrinB3-Fc (R&D Systems) (each at 13 nM), were incubated with 40 nM ¹²⁵I-FGF2 (specific activity: ˜50 μCi/μg, MP Biochem.) with or without the indicated concentrations of cold FGF2 or insulin for 2 h at room temperature in PBS. Cross-linking was initiated by adding BS³ (bis[sulfosuccinimidyl]suberate, Pierce, Rockford, Ill.) to a final concentration of 2 mM and incubating for 30 min at room temperature. The reaction was stopped by adding 2 μl of 1 M Tris-HCl (pH 7.4) and protein complexes were analyzed by SDS-PAGE. Gels were subsequently fixed, dried, and subjected to autoradiography.

PC12 Cell Cultures, Neurite Outgrowth Assay, and Analysis of FGF2 Signaling

Rat phaeochromocytoma PC12 cells were maintained in DMEM supplemented with 10% FBS and 5% horse serum. Clonal cell lines were obtained by transfection of PC12 cells with the pcDNA3.0 plasmid containing full-length rat NgR1 cDNA followed by selection with G418 over several weeks. Clonal cell lines expressing NgR1 were isolated by limiting dilution and identified by anti-NgR1 immunocytochemistry.

For differentiation experiments, PC12 cells were plated at low density (10,000 cells/well of 24 well plate) on poly-lysine coated plate. After overnight culture, cells were kept in low serum media (DMEM supplemented with 0.5% FBS and 0.25% horse serum). After 24 hours growth factors were added to the cells in low serum medium for 4 days. Fresh growth factor was added after two days. On the fourth day, cells were fixed in 4% paraformaldehyde in PBS and immunostained for cell surface NgR1 using anti-NgR1 antibody under non-permeabilizing conditions and then permeabilized with 0.1% TritonX-100 and double stained using anti-E-tubulin III antibody.

For analysis of activation of ERK1/2 and MEK, PC12 cells were plated on poly-lysine coated 24-well plates at 50,000 cells/well. Cells were serum-starved in DMEM with 0.1% FBS for 24 hr and were treated with FGF1 (R&D Systems), FGF2, or EGF (Peprotech, Rocky Hill, N.J.) for 5 min at room temperature and lysed in 2xLaemmli sample buffer. Cell lysates were subjected to Western blotting using a set of anti-p44/42 ERK and anti-phospho-specific p44/42 ERK antibodies, a set of anti-MEK and anti-phospho-specific MEK antibodies (Cell Signaling, Danvers, Mass.). Membranes were stripped and reprobed with anti-actin antibody. PC12 cells were transfected with caRas (UMR cDNA Resource Center, Rolla, Mo.), FGFR1, and FGFR3 to examine FGF signaling in PC12^{NgR1} and control cells using the Amaxa Biosystems (Gaithersburg, Md.) nucleofection technology. Cells were treated with FGF2 and cell lysates were prepared as described herein. For analysis of FRS2α activation, cells were treated with growth factors as described herein. Membrane fractions were prepared by homogenizing cells in 50 mM Tris pH 7.6, 150 mM NaCl, 5% sucrose and protease inhibitor mixture. Nuclei and cell debris were removed by centrifugation at 500 ug for 10 min at 4° C. The resulting supernatant was centrifuged at 110,000 μg for 45 min at 4° C. The membrane pellet was solubilized in RIPA buffer (50 mM Tris-HCl, pH 8.0, 150 mM NaCl, 1% NP-40, 0.5% sodium deoxycholate, 0.1% SDS and protease inhibitor mixture). Insoluble material was removed by centrifugation at 14,000 μg for 10 min at 4° C. The solubilized membrane was subjected to Western blotting using anti-FRS2α (Santa Cruz Biotechnology, Santa Cruz, Calif.) and anti-phospho-FRS2α (Tyr⁴³⁶) (Cell Signaling) antibodies.

Transient Expression of NgR1 in PC12 Cells

Amaxa nucleofection technology was used to transiently express NgR1 or eGFP in PC12 cells. Following transfection, cells were incubated in high serum culture medium overnight and changed to low serum medium for 1 day. Cells were then treated with FGF2 (25 ng/ml) for 3 days prior to fixation. Cells successfully transfected were identified by GFP expression or anti-NgR1 immunofluorescence and TuJ1, as described (Venkatesh et al., 2005). Consistent with our results obtained with PC12^{NgR1} cell lines, transient expression of NgR1 but not GFP results in a statistically significant reduction of cells that undergo FGF2-elicited differentiation, as assessed by quantification of cells bearing neurites longer than two cell body diameters (p<0.05), Students t-test.

Synaptic Fractions from Adult Rat Hippocampus

Preparation of Synaptosomes and Subcellular Fractionations were Carried Out as described (Phillips et al., 2001). Fractions were analyzed with antibodies specific for NgR1, OMgp (R&D Systems), FGFR1 (Santa Cruz), Syndecan-3 (A. Oohira), Syntaxin 1A (Stressgen, Ann Arbor, Mich.), PSD-95 and NR1 (Upstate, Lake Placid, N.Y.), and synaptophysin (Sigma, St. Louis, Mo.).

Electrophysiology

Wild-type C57BL/6 or NgR1^{-/-} mice (Zheng et al., PNAS 102:1205-10, 2005) between 6-8 weeks of age were decapitated; the brains were quickly removed and immediately placed in ice-cold artificial cerebrospinal fluid (ACSF: 125 mM NaCl, 1.25 mM NaH₂PO₄, 25 mM Glucose, 25 mM NaHCO₃, 2.5 mM CaCl₂, 1.3 mM MgCl₂, 2.5 mM KCl saturated with 95% O₂/5% CO₂). Sagittal hippocampal slices (400 μm) were cut on a vibrating microtome and maintained in oxygenated ACSF at room temperature for at least 1 hr. For recording, the slices were transferred to an immersion chamber, continuously perfused at 3 ml/min with oxygenated ACSF, and maintained at 32° C.±0.5° C.

fEPSPs were recorded from the CA1 stratum radiatum region. Briefly, a platinum/iridium concentric bipolar electrode (FHC Inc, Bowdoinham ME) was used to stimulate Schaffer collateral afferents. Recordings were done with glass microelectrodes filled with ACSF (pipette resistance ˜0.3-0.4 MΩ: Paired pulse facilitation (PPF) was assessed at interpulse intervals of 25, 50, 100, 200, 300, 400, and 500 ms. Slices were monitored with stimuli consisting of constant current pulses of 0.1 ms duration at 0.067 Hz. After a stable baseline of <45 min (˜1 mV), LTP was induced at >50% of maximal amplitude by high frequency stimulation (HFS, 100 Hz, 1 s duration, 2 trains, interval 10 s) as described previously (Meng et al., 2003). Slices that did not show a stable baseline for at least 30 mins prior to stimulation were discarded. Recorded potentials were filtered at 3 kHz, digitized at 12.5 kHz, and stored for later analysis. fEPSPs were analyzed by fitting 3rd order polynomials to the sweeps, first to measure the peak, and then to measure the slope at the 50% amplitude point. All fits were monitored visually on the oscilloscope screen. Data were normalized to the baseline average. For local application of drugs through the recording electrode, see below. Data were analyzed statistically using Sigma Stat 3.5 one way ANOVA, post-hoc Tukey's test.

Histology and Dendritic Spine Analysis

Brain samples of mice at postnatal days 71-104 were removed and submerged in 10 ml of Golgi-Cox solution and processed as previously described (Gibb and Kolb, 1998). Spine density analysis was performed blindly of genotyping. In area CA1 of the dorsal hippocampus of each animal, apical dendrites of the stratum radiatum of 8-15 randomly selected pyramidal neurons were examined. These neurons were required to have no breaks in staining along the dendrites. Measurement occurred at least 50 μm away from the soma on secondary and tertiary branches. Approximately 30 dendritic branches (˜10 μm each) were analyzed from each brain. All analysis was done by a single rater on an Olympus BX60FS microscope at an optical magnification of 1000× magnification and oil immersion. For spine morphology, spines were assigned the morphological category (Harris et al., 1992; see below) that most resembled the shape of the spine. Spine density was calculated by dividing the number of spines on a segment by the length of the segment and was expressed as the number of spines per 10 μm of dendritic length. Means for the density of spines were analyzed using ANOVA.

For electron microscopy, 6 week old wild-type (n=2) and NgR1 mutants (n=3) were perfused in ice cold parafomaldehyde (2%) and glutaraldehyde (2%) solution. Coronal slices were cut and brains were post-fixed in 1.0% Osmium peroxide buffered in 0.1M sodium cacodylate for one hour. Brain slices were rinsed in the same buffer (3 times) and distilled water, placed into 50% ethanol and stained in a solution of 0.5% uranyl acetate/50% ethanol overnight at 4° C. The sections were thoroughly rinsed in 50% ethanol and dehydrated in a graded series of ethanol to 100%, transferred to propylene oxide, and embedded with the epoxy resin mixture of EPON/araldite. The blocks were polymerized two days at 70° C., sectioned with glass knives at 1 micron and stained with Toluidine blue to determine the area of hippocampus to thin section with a diamond knife onto 200 mesh grids. The grids were stained sequentially 10 minutes each in uranyl acetate and lead citrate. A Hitachi 7100 transmission electron microscope with attached MegaView III (Olympus Soft Imaging Systems, Lakewood, Colo.) digital camera was used to generate images at 15,000 magnification. Approximately 25 electron micrographs were taken of the CA1 dendritic field and analyzed per animal. Digital images were converted to Tiff files and later processed in Adobe Photoshop 7.0.1. Clearly discernible synapses consisting of presynaptic terminal with synaptic vesicles and a spine with a defined postsynaptic density were counted. Perforated synaptic junctions and multiple synapses of one spine with more than one axon terminal were treated as one synapse. Mean synapse densities were calculated for each animal and combined for each group. A Student's t test was used to measure significance.

Biochemical Enrichment of sNgR1 Binding Activity

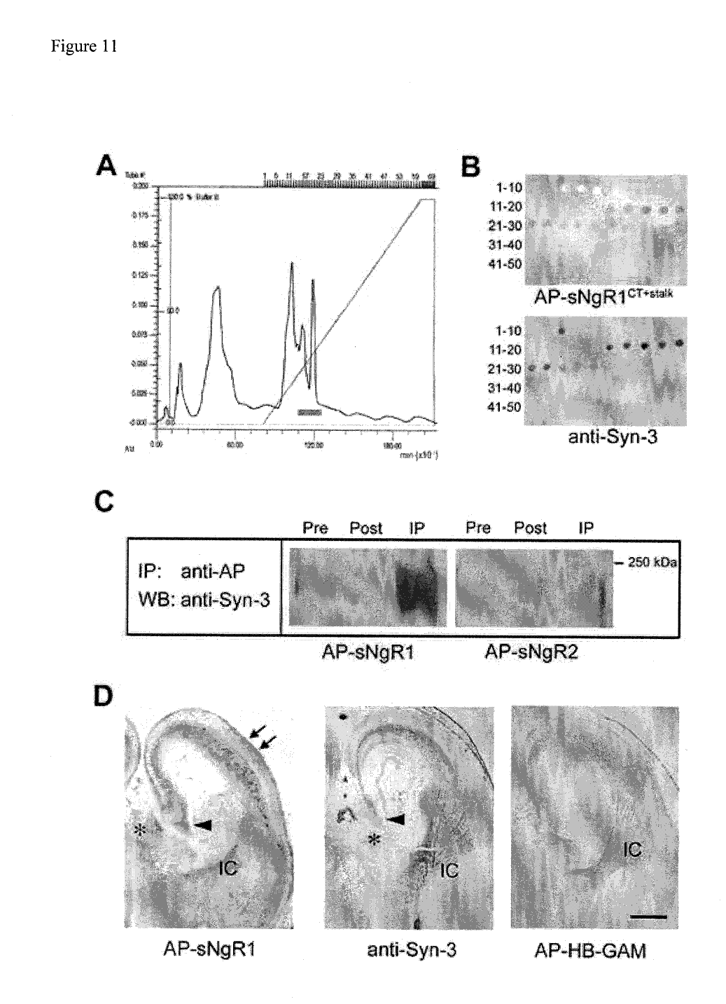

To identify the proteoglycans involved in AP-sNgR1 binding, postnatal day 7 rat brains were homogenized in extraction buffer (50 mM TRIS pH 8.0, 150 mM NaCl, 60 mM CHAPS, 1 mM EDTA and protease inhibitor cocktail (Sigma)) and incubated for 30 minutes on ice. Extracts were cleared by two high-speed spins at 167,000×g for 2 hr at 4° C. The supernatant was collected and centrifuged a 420,000×g for 1 hr at 4° C. The extract was then filtered through a 0.22 μm sterile filter. Proteoglycans from crude brain extracts (20 mg total at 1 mg/ml) were loaded on to a mono-Q anion-exchange column (BioRad, Hercules, Calif.) column equilibrated in buffer A (0.1% CHAPS (w/v), 50 mM TRIS buffer pH 8.0) and eluted in a linear salt gradient from 0-2M NaCl in 0.1% CHAPS, 50 mM TRIS buffer pH 8.0 (60 mls volume at a rate of 1 ml/min) using a duo-flow FPLC system (BioRad) (FIG. 11A). Proteins eluting at increasing salt concentrations were spotted onto a nitrocellulose membrane (10 μl/spot) and subsequently, heated to 65 degrees for 2 hours. Following, membranes were blocked in 0.5% dry milk/TBST for 1 hr at room temperature and probed for AP-sNgR1CT+stalk binding (see FIG. 11B). Strongest binding activity was found in fractions 16-25, eluting between ˜0.4-0.7 M NaCl. Fractions that showed strongest binding of AP-sNgR1^{CT+stalk} were pooled, concentrated by ultra-filtration (Centricon 10,000 Da MWCO; Millipore, Billerica, Mass.), and further fractionated via size exclusion over a Sephadex-G250 column (Amersham, Piscataway, N.J.). AP-sNgR1^{CT+stalk} bound robustly to molecules eluting at ˜150-300 kDa. Fractions that support AP-sNgR1^{CT+stalk} binding were spotted onto a nitrocellulose membrane and assayed for the presence of neural heparan sulfate proteoglycans, including members of the syndecan and glypican families and agrin. To examine whether AP-sNgR1CT+stalk forms a complex with syndecan-3, dialyzed protein fractions eluting between 0.5-0.7 M NaCl and probed positive for AP-sNgR1^{CT+stalk} binding were incubated with AP-sNgR1^{CT+stalk} and immunoprecipitated with an anti-AP serum (American Research Products, Belmont, Mass.). The immune-complex was assayed for the presence of syndecan-3 by Western blot analysis using polyclonal rabbit anti-syndecan-3 serum (A. Oohira; 1:1000 dilution). A prominent band with a molecular weight of ˜150-200-kDa was pulled down with AP-sNgR1^{CT+stalk}. As a control, AP-sNgR2^{CT+stalk} was used and no enrichment of neural syndecan-3 was found in the precipitate (FIG. 11C). Together these results show that neural syndecan-3 is a HSPG binding partner of NgR1 but not NgR2.

Focal Drug Application

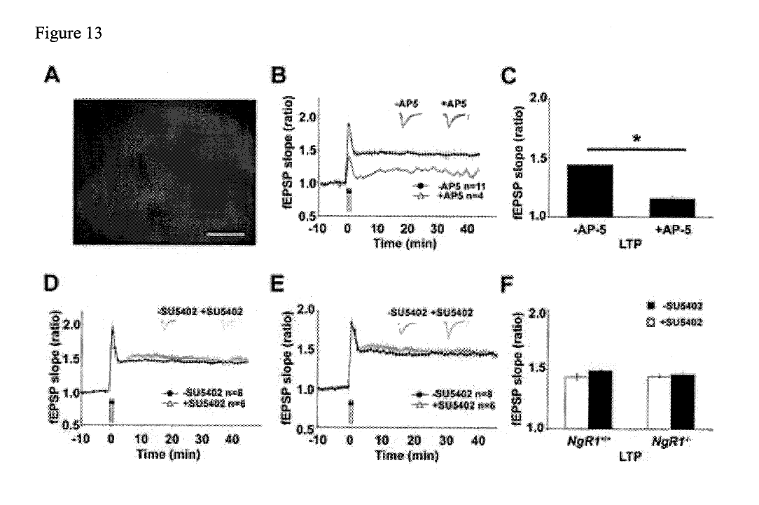

For local application, drugs, FGF2 and FGF8 (Peprotech), OMgp-Fc (R&D Systems) were diluted in ACSF to a final concentration of 10 μg/ml, loaded in the recording pipette, and delivered to CA1 directly through the recording pipette (Castro-Alamancos et al., 1995; Feldman, 2000; Pesavento et al., 2000). To visualize the diffusion and tissue distribution of focally applied molecules we used Texas-red conjugated dextran MW=10 kDa (Molecular Probes (Invitrogen), Carlsbad, Calif.) and pictures of the CA1 region were taken after 45 min using an Nikon Diaphot (FIG. 13A). In addition, the NMDA receptor antagonist AP5 (D,L-2-amino-5-phospho-novalerate) was locally applied through the recording electrode (100M) significantly suppressed LTP, demonstrating successful drug delivery to the CA1 region in acute hippocampal slices (FIG. 13B,C).

Identification and Characterization of a Lectin Activity Associated with Soluble NgR1 and NgR3, but not NgR2

Binding results of AP-tagged Nogo receptor fusion proteins to embryonic and neonatal mouse brain tissue sections and transiently transfected COS-7 cells were assayed. Semi-quantitative analysis of the binding strength is shown in Table S1: (+++) very strong binding; (++) moderately strong binding; (+) weak binding but clearly detectable; (−) no binding above background. Binding to brain tissue sections of p75 (exonIII), p75 (exonIV), and NgR1 mutants is comparable to wild-type brain tissue. Furthermore, binding studies with GalNAcT^{-/-} and GD3 S^{-/-} brain tissue revealed that gangliosides are dispensable for AP-sNgR1^{CT+stalk} or AP-sNgR3^{CT+stalk} binding. Binding studies to recombinant proteins expressed on the surface of COS-7 cells, did not reveal any interactions with previously identified components of the Nogo receptor complex. Binding is highly resistant to heat treatment and preincubation with trypsin. In the presence of heparinase or exogenously added heparin, a highly sulfated form of heparan sulfate (HS), binding of AP-sNgR1^{CT+stalk} and AP-sNgR3^{CT+stalk} to brain tissue section is greatly reduced. Taken together, these studies suggest that NgR1 and NgR3, but not NgR2, interact with neural glycans including HS-GAG side chains of axon associated HSPG(s).

TABLE S1
Identification and characterization of a lectin activity associated with soluble NgR1 and NgR3

| soluble receptor binding to | AP-sNgR1^{CT+stalk} | AP-sNgR2^{CT+stalk} | AP-sNgR3^{CT+stalk} | MAG-Fc |
|---|---|---|---|---|
| E18 brain sections | +++ | − | +++ | N/A |
| P3 brain sections | +++ | − | +++ | N/A |
| E18 brain p75^{-/-} (exonIII) | +++ | − | +++ | N/A |
| P3 brain p75^{-/-} (exonIV) | +++ | − | +++ | N/A |
| E18 brain NgR1^{-/-} | +++ | − | +++ | N/A |
| P3 brain GlacNAcT^{-/-} | +++ | − | +++ | N/A |
| P3 brain GDS3^{-/-} | +++ | − | +++ | N/A |
| p75 in COS cells | − | − | − | − |
| Troy in COS cells | − | − | − | − |
| Lingo-1 in COS cells | − | − | − | − |
| NgR1 in COS cells | − | − | − | + |
| NgR2 in COS cells | − | − | − | +++ |
| NgR3 in COS cells | − | − | − | − |
| L-MAG in COS cells | − | − | − | ++ |
| OMgp in COS cells | − | − | − | − |
| Nogo66-Npn1 in COS cells | − | − | − | − |
| E18 brain + heat (75° C.) | +++ | − | +++ | N/A |
| E18 brain + trypsin | +++ | − | +++ | N/A |
| E18 brain + Hep'aseIII | + | − | + | N/A |
| E18 brain + heparin | − | − | + | N/A |

NgR1 and NgR3 Associate with Neural Proteoglycans

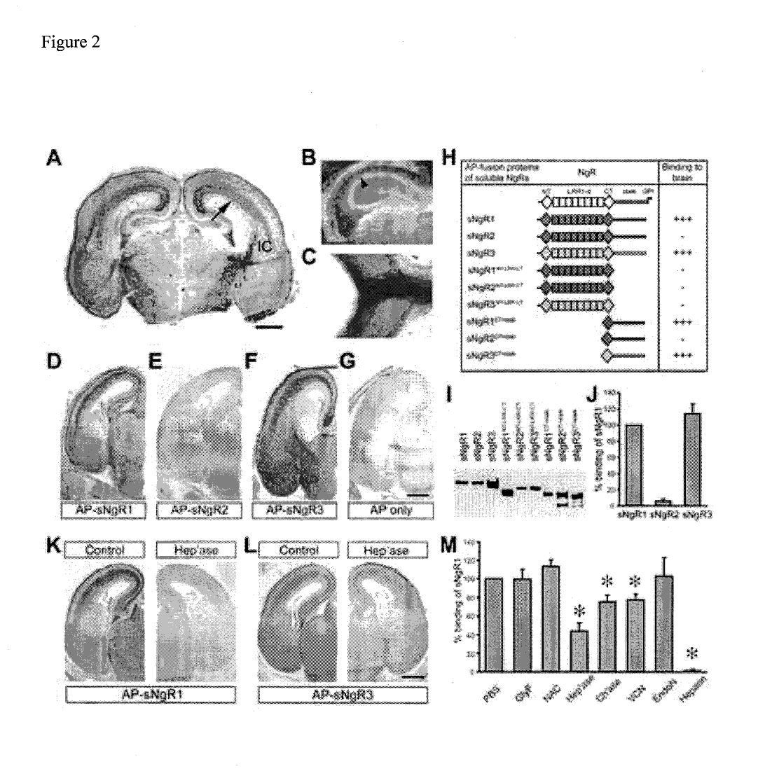

Nogo receptor family members are GPI-anchored proteins composed of a tandem array of eight LRRs flanked by cysteine-rich N-terminal (NT) and C-terminal (CT) cap domains. The NT-LRRs-CT domains adopt a curved solenoid fold and are connected through a C-terminal stalk to the plasma membrane. The NgR1 NT-LRR-CT cluster (NgR310) is sufficient to support binding of the myelin inhibitors Nogo-A, MAG, and OMgp (Barton et al., EMBO J. 22:3291-3302, 2003; Fournier et al., J. Neurosci. 22:8876-8883, 2002). A membrane bound deletion mutant of NgR1 that lacks the stalk region has dominant-negative activity, indicating an important role for the NgR1 stalk in signaling inhibitory neuronal responses (Wang et al., Nature 417:941-44, 2002). To begin exploring how the stalk region participates in NgR1 signaling, soluble alkaline phosphatase (AP) tagged fusion proteins of NgR1 comprised of various deletion mutants were used and their ability to bind to embryonic and neonatal brain tissue sections was analyzed in situ (FIG. 2). Experiments with the ectodomain of NgR1 (AP-sNgR1) and a deletion mutant comprised of the CT cap domain and stalk region (AP-sNgR1^{CT+stalk}) revealed a complex labeling pattern. Both fusion proteins show very similar binding to a number of CNS fiber tracts, including the internal capsule, hippocampal alveus, fimbria-fornix and optic nerve (FIG. 2A-C). A similar, yet distinct labeling pattern was observed with the ectodomain of NgR3 (AP-sNgR3) and the deletion construct AP-NgR3^{CT+stalk} (FIG. 2). Under similar conditions, AP-sNgR1^{NT-LRR-CT} and AP-sNgR3^{NT-LRR-CT} do not bind to brain tissue sections. Interestingly, fusion proteins of NgR2, including AP-sNgR2 and AP-NgR2^{CT+stalk} show no binding to brain tissue (FIG. 2D-J).

Experiments summarized in Table S1 revealed that neither AP-sNgR1CT+stalk nor AP-NgR3CT+stalk binds to previously identified components of the NgR1 receptor complex. Initial studies aimed at the molecular characterization of neural binding partners for AP-sNgR1^{CT+stalk} and AP-sNgR3^{CT+stalk} showed that the interaction is highly resistant to heat and protease treatment. To examine whether Nogo receptors associate with neural glycoconjugates, brain tissue sections were preincubated with enzymes acting on various glycoconjugates. Digestion with heparinase III and to a lesser extent with chondroitinase ABC or V. cholerae neuraminidase, leads to a significant reduction in binding of AP-sNgR1^{CT+stalk} or AP-sNgR3^{CT+stalk} to brain tissue sections. In marked contrast, N-acetylglucosaminidase, glycopeptidase F, or endoneuraminidase-N incubation had no effect on binding (FIG. 2K-M). This shows that neural glycosaminoglycans (GAGs) and, to a lesser extent, terminal sialic acids participate in sNgR1 and sNgR3 binding to brain tissue.

To identify the heparan sulfate carrier protein(s) involved in AP-sNgR1^{CT+stalk} binding, proteoglycans from crude neonatal brain extracts were enriched by anion exchange chromatography. Proteins eluting at increasing salt concentration were spotted on a nitrocellulose membrane and probed for AP-sNgR1^{CT+stalk} binding (FIG. 11A,B). A close overlap between AP-sNgR1^{CT+stalk} binding and syndecan-3 but not glypican-1, -4, -5 or agrin immunoreactivity was found (FIG. 11B). Combined fractions that support AP-sNgR1^{CT+stalk} binding were incubated with AP-sNgR1^{CT+stalk}, precipitated with anti-AP, and the immunocomplex was assayed for the presence of syndecan-3. A prominent band with a molecular weight of ˜150-200 kDa was pulled down by AP-sNgR1^{CT+stalk} but not by AP-sNgR2^{CT+stalk} (FIG. 11C), showing neural syndecan-3 is an HSPG binding partner of NgR1 but not NgR2. Consistent with the idea that neuronal syndecan-3 supports AP-sNgR1 binding, a close overlap existed between anti-syndecan-3 labeling (Hsueh and Sheng, J. Neurosci. 19:7415-7425, 1999) and the AP-sNgR1 binding pattern. Moreover, AP-tagged pleiotrophin/HBGAM, a previously identified syndecan-3 ligand (Kinnunen et al., Eru. J. Neurosci. 11:491-502, 1999), and AP-sNgR1 showed strikingly similar binding patterns to E18 brain tissue sections (FIG. 11D).

NgR1 Supports Binding of Select Members of the FGF Family

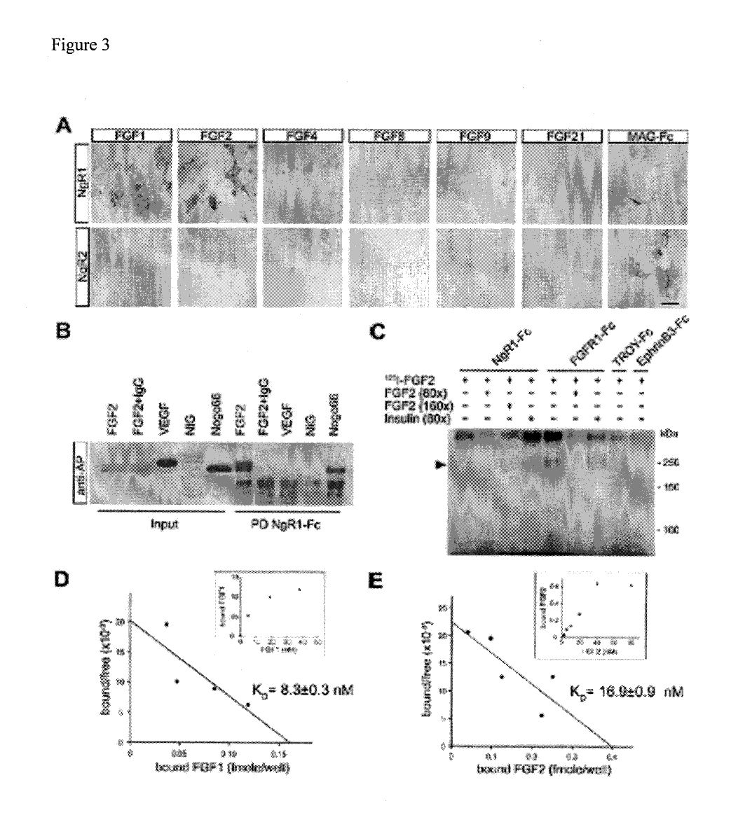

Syndecans bind to a variety of growth factors and molecules of the extracellular matrix via their GAG chains and protein cores (Lopes et al., Braz. J. Med. Biol. 39:157-67, 2006). Experiments were performed to determine whether previously identified syndecan binding partners also interacted with NgR1. In COS-7 cells, NgR1 does not support binding of AP-VEGF₁₆₅ or AP-HBGAM. Remarkably, however, the syndecan-3 ligand FGF2 (Chemousov and Carey, J. Biol. Chem. 268:16810-4, 1993) binds strongly to NgR1 but not to NgR2 (FIG. 3A). To explore the possibility that other members of the FGF family bind to Nogo receptors, AP-FGF1, AP-FGF4, AP-FGF8, AP-FGF9 and AP-FGF21 fusion proteins were generated. Similar to FGF2, FGF1 binds robustly to NgR1 but not to NgR2. A much weaker association was found between NgR1 and FGF4. Under similar conditions, no association between NgR1, and FGF8, FGF9, or FGF21 was detected (FIG. 3A).

To address whether NgR1 interacts directly with FGF2, affinity precipitation experiments were performed. NgR1-Fc selectively and specifically forms a complex with AP-FGF2 and AP-Nogo66 but not with AP-VEGF₁₆₅ or AP-NiG, an inhibitory fragment of Amino-Nogo (FIG. 3B). In a parallel approach, ¹²⁵I-FGF2 to independently demonstrate the specificity of the NgR1-FGF2 association. ¹²⁵I-FGF2 can be crosslinked selectively to NgR1-Fc and FGFR1-Fc but not to TROY-Fc or ephrinB3-Fc. Moreover, formation of the ¹²⁵I-FGF2 complex with NgR1-Fc or FGFR1-Fc is specific and efficiently competed by excess unlabeled FGF2 but not insulin (FIG. 3C). The complexes of ¹²⁵I-FGF2:NgR1-Fc and ¹²⁵I-FGF2:FGFR1-Fc run at apparent molecular weights of 220- and 250-kDa and higher molecular weight complexes containing ¹²⁵I-FGF2 are detected as well (FIG. 3C). Scatchard plot analysis of AP-FGF1 and AP-FGF2 binding to NgR1 expressed on the surface of COS-7 cells revealed dissociation constants (K_{D}s) of 8.3±0.3 nM (FGF1) and 16.9±0.9 nM (FGF2) (FIG. 3D,E). Taken together, novel and direct interactions between NgR1 and select members of the FGF family were identified.

NgR1 is a Negative Regulator of FGF1 and FGF2 Signaling

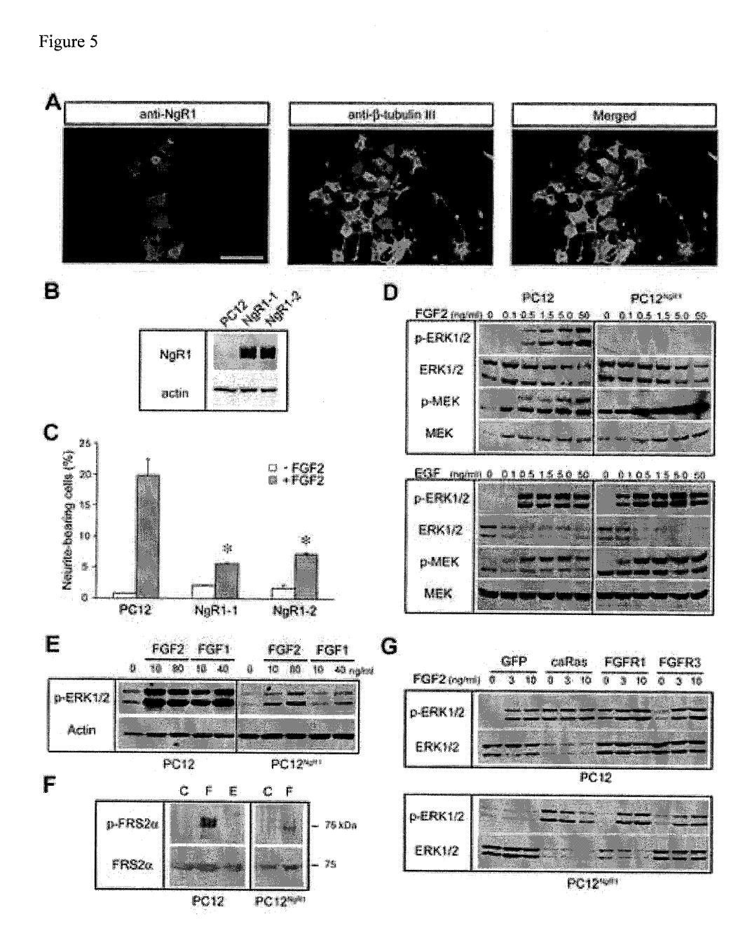

Next, to address the functional significance of the NgR1-FGF2 association, PC12 cell lines stably expressing NgR1 (PC12^{NgR1}) were generated to determine whether ectopic NgR1 modulates FGF2-elicited PC12 cell differentiation (FIG. 5A,B). Control PC12 cells express very low levels of endogenous NgR1 and, in the presence of FGF2, cells extend neurite-like processes. Double immunofluorescence labeling of a heterogeneous cell population with anti-NgR1 and anti-E-tubulin III antibodies revealed that PC12 cells overexpressing NgR1 selectively fail to extend neurite-like processes when treated with 25 ng/ml FGF2 (FIG. 5A). This shows that ectopic NgR1 inhibits FGF2-elicited cell differentiation. Quantification of FGF2-elicited process outgrowth revealed a significant decrease in the percentage of cells with processes longer than two cell bodies in diameter. Two independent cell lines of PC12^{NgR1} (NgR1-1; 5.6±0.1 and NgR1-2; 7.2±0.2%) showed significantly fewer cells with long neurites (p<0.05) when compared to control PC12 cells (19.8±2.6%) (FIG. 5C). As an independent control, transient expression of NgR1, but not eGFP, in PC12 cells was shown to result in a similar inhibition of FGF2-elicited differentiation. Importantly, ectopic NgR1 does not influence cell viability or mitotic rate, as assessed by cell counts over several passages.

In PC12 cells, FGF2 treatment leads to prolonged activation of the p44 (ERK1) and p42 (ERK2) MAP kinases through the FGFR-FRS2-ras-MAPK pathway, a necessary step to induce cellular differentiation (Kouhara et al., 1997). To explore whether NgR1 expression regulates FGF2-elicited activation of the ERK1/2 MAP-kinase pathway and its upstream dual specific kinase MEK, control PC12 and PC12^{NgR1} cells were treated with increasing doses of FGF2 and cell lysates were analyzed by immunoblotting with phospho-specific anti-ERK1/2 and anti-MEK antibodies. In control PC12 cells, FGF2 leads to a rapid and dose-dependent activation of the MEK-ERK1/2 pathway. In marked contrast, FGF2 fails to elicit MEK or ERK1/2 activation in PC12^{NgR1} cells at any concentration up to 50 ng/ml (FIG. 5D). In the presence of EGF, however, both control PC12 and PC12^{NgR1} cells show robust activation of the MEK-ERK1/2 pathway, suggesting that NgR1 selectively blocks FGF2, but not EGF-elicited activation of ERK1/2 pathway. Similar to FGF2, FGF1-induced activation of ERK1/2 is strongly inhibited in PC12^{NgR1} cells (FIG. 5E). The docking proteins FRSαβ are major regulators of FGFR signaling (Kouhara et al., Cell 89:693-702, 1997), and in PC12^{NgR1} cells FGF2-induced activation of FRS2α is markedly decreased compared to controls showing that NgR1-mediated regulation of FGF signaling is upstream of FRS2 (FIG. 5F).

To more directly address at which level NgR1 blocks the FGFR/MAP-kinase pathway, several rescue experiments were performed. Following transient transfection of constitutively active H-ras (rasV12), but not eGFP, PC12^{NgR1} shows robust and FGF2-independent activation of ERK1/2. Furthermore, transient expression of FGFR1 or FGFR3 in PC12^{NgR1} cells is sufficient to allow FGF2-dependent activation of ERK1/2 (FIG. 5G). Together these findings show that NgR1 inhibits FGFR signaling at the receptor level.

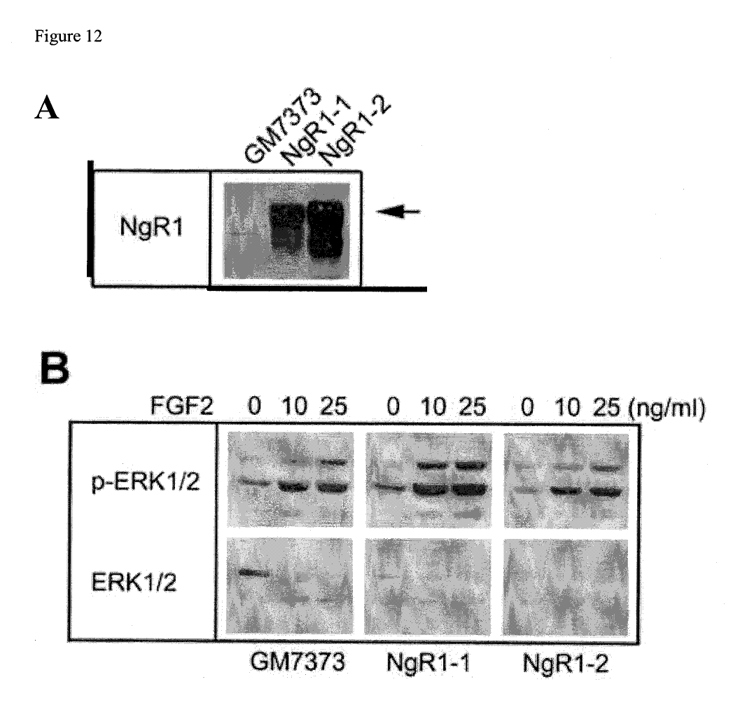

To expand on these observations, experiments were performed to determine whether NgR1 inhibits FGF2-induced activation of the ERK1/2 MAP-kinase pathway in cells that respond to FGF2 not by differentiation but by proliferation. Interestingly, ectopic expression of NgR1 in GM7373 cells, a cell line derived from bovine endothelial cells, does not attenuate FGF2-induced activation of ERK1/2 (FIG. 12). This shows that NgR1 attenuates FGF2-elicited activation of the ERK1/2 MAP kinase pathway in a cell-type specific manner.

NgR1 Regulates Hippocampal LTP in an FGF2-Dependent Manner

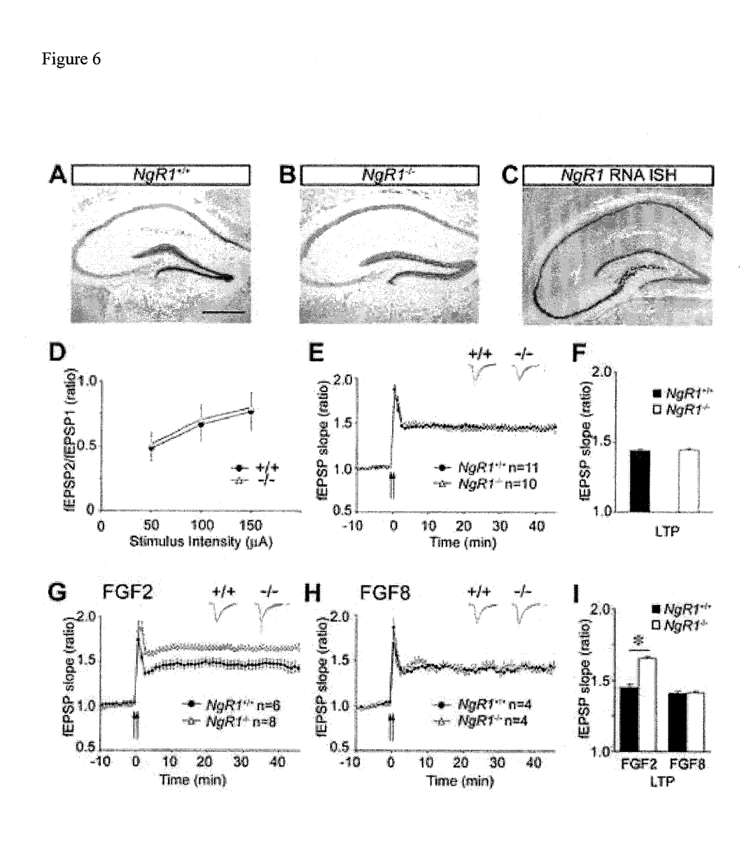

Experiments were performed to determine whether NgR1 influences plasticity at glutamatergic synapses. Niss1 staining of wild-type and NgR1 null adult hippocampal sections revealed no differences at the gross anatomical level (FIG. 6A,B). In situ hybridization confirmed strong expression of NgR1 in CA3 and CA1 pyramidal neurons and somewhat less intense expression in dentate granule cells (FIG. 6C).

To study the role of NgR1 in synaptic function, electrophysiological experiments were conducted in acute hippocampal slices of 6-8 week old wild-type and NgR1 mutant mice. Basal transmission at Schaffer collateral-CA1 synapses was unaltered between slices prepared from wild-type (n=8 slices/5 animals) and NgR1 null (n=10 slices/4 animals) mice as assessed by input/output (I/O) curves. I/O curves were constructed using three stimulus levels and no changes in single stimulus-evoked responses were observed between the two genotypes, suggesting that lack of NgR1 does not alter basal synaptic transmission (FIG. 6D). Mice lacking syndecan-3 exhibit an increased level of LTP in area CA1 (Kaksonen et al., Mol. Cell. Neurosci. 21:158-72, 2002). To examine whether loss of NgR1 has an effect on long-term synaptic plasticity, LTP was assessed at the Schaffer collateral-CA1 synapses in acute hippocampal slices. To induce LTP, two trains of high frequency stimulation (HFS; 100 Hz, 1 sec, separated by a 10 second interval) were applied. LTP in wild-type (mean fEPSP=144±0.9% of baseline n=11 slices/9 animals) and NgR1-deficient slices (mean fEPSP=145±1.1%, n=10 slices/9 animals) was robust and indistinguishable, showing that induction and consolidation of LTP in CA1 neurons is NgR1-independent (FIG. 6E,F). Furthermore, the data show that proper development and patterning of the hippocampal CA3-CA1 circuitry does not require NgR1 function.

Next synaptic function in NgR1 mutant slices was assessed to determine whether it is altered in the presence of FGF2. LTP experiments were repeated in the presence of FGF2, locally applied to the CA1 dendritic field through the recording pipette as described above. In wild-type hippocampal slices, focal application of FGF2 (10 μg/ml) did not result in a significant alteration of HFS induced LTP (mean fEPSP=139±3.0% of baseline, n=6 slices/4 animals) (FIG. 6G,I) as compared to no ligand controls (FIG. 6E,F). In marked contrast, NgR1 null hippocampal slices showed an FGF2-dependent enhancement of LTP (mean fEPSP=165±1.1%, n=8 slices/6 animals; p<0.001) (FIG. 6G,I). To ask whether the observed increase in LTP is specific for FGF2, experiments were repeated with FGF8, a FGF family member that does not bind to NgR1. As shown in FIGS. 6H and 6I, in the presence of locally applied FGF8 (10 μg/ml), LTP is not enhanced in NgR1 mutants (fEPSP=141±1.2%; n=4 slices/3 animals) or wild-type slices (fEPSP=141±1.4%; n=4 slices/3 animals) and is comparable to no ligand controls (FIG. 6E). These results indicate that the observed ligand-dependent increase in LTP in NgR1 mutants is FGF2-specific. During LTP NgR1 negatively regulates FGF2 signaling at the CA3-CA1 synapse, showing that NgR1 is involved in activity-dependent regulation of synaptic strength in the adult hippocampus.

Hippocampal NgR1 is Localized to Synapses

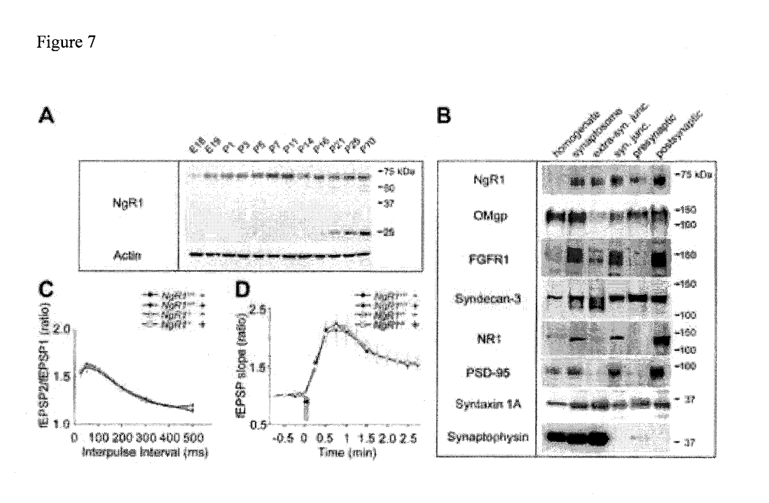

During late embryonic development, hippocampal NgR1 expression increases rapidly, peaks in the second postnatal week, and remains high throughout adulthood (FIG. 7A). Interestingly, a significant portion of NgR1 undergoes proteolytic processing in an age-dependent manner. A proteolytic fragment of 23-kDa reacts with an antibody directed against the C-terminal portion of NgR1 and increases in abundance in older animals (FIG. 7A). To determine whether NgR1 is present at synapses, its distribution was examined in synaptosomal fractions generated from adult rat hippocampus (Phillips et al., Neuron 32:63-77, 2001). NgR1 was enriched in synaptosomes and localized preferentially to PSD-95 and NR1-positive postsynaptic density fractions (FIG. 7B). Similar to NgR1, OMgp and FGFR1 were present in synaptosomes, synaptic junctions, and were enriched in postsynaptic density fractions. Syndecan-3 was enriched in synaptosomes and was abundantly found in extra-synaptic, pre- and post-synaptic fractions (FIG. 7B).

Two Forms of Short-Term Plasticity, PPF and PTP, are Unaltered in NgR1 Mutants

To address whether NgR1 functions pre- or post-synaptically, two pre-synaptically driven forms of short-term plasticity, paired-pulse facilitation (PPF) and post-tetanic potentiation (PTP), were assessed at Schaffer collateral-CA1 excitatory synapses in acute hippocampal slices. PPF measures transient enhancement of neurotransmitter release induced by two closely spaced stimuli due to accumulation of intracellular calcium (Schulz et al., 1994). We measured PPF at interstimulus intervals of 25-500 msec and facilitation was observed at all intervals tested. There was no difference detected in PPF between wild-type (n=11 slices/7 animals) and NgR1 mutants (n=10 slices/6 animals). Further, no significant change in PPF was found in either animal group when FGF2 was applied (wild-type n=11 slices/7 animals; NgR1 mutant n=8 slices/4 animals) (FIG. 7C). PTP, thought to be caused by enhanced presynaptic transmitter release due to loading of the presynaptic terminal with calcium following high frequency stimulation, was also assessed (Zucker and Regehr, 2002). PTP measured over a 0.25-5 min interval following tetanization was not altered between wild-type (n=3 slices/2 animals, no FGF2; and n=3 slices/2 animals, with FGF2) and NgR1 mutants (n=4 slices/2 animals, no FGF2; and n=5 slices/2 animals, with FGF2) (FIG. 7D). Taken together, these electrophysiological assays argue against a presynaptic role of NgR1, either in the presence or absence of FGF2.

FGFR Kinase Activity is Necessary for Enhanced LTP in NgR1 Mutants

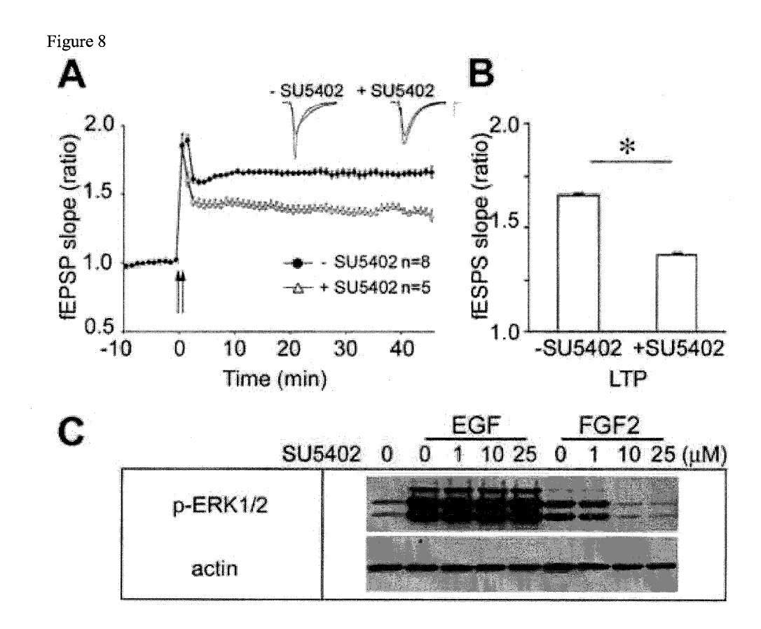

Biochemical studies found an interaction between NgR1 and syndecan-3 (FIG. 11). Syndecans are transmembrane HSPGs that interact via the cytoplasmic tail with src-kinase, CASK, and syntenin (Lopes et al., Braz. J. Med. Biol. 39:157-67, 2006). Reorganization of the spine actin cytoskeleton is important for LTP consolidation (Krucker et al., PNAS 97:6856-6861, 2000) and in the presence of FGF2, spine actin polymerization and LTP consolidation could, in principle, be regulated through syndecan-3 rather than FGFR signaling. To test whether FGFR signaling participates in FGF2-elicited enhancement of LTP in NgR1 mutants, experiments were repeated in the presence of the FGFR kinase inhibitor SU5402 (Mohammadi et al., Science 276:955-60, 1997). Exposure to SU5402 (1 mM) in the absence of FGF2 does not alter HFS-induced LTP in either wild-type or NgR1 null hippocampal slices as compared to vehicle treated slices (FIG. 13D-F). However, when both were present, SU5402 blocked the FGF2-induced enhancement of LTP in NgR1 null slices (n=5 slices/3 animals), thus, indicating that FGFR kinase activity is necessary for the expression of the FGF2-dependent enhancement of LTP in NgR1 mutants (FIG. 8A,B). As a control for the specificity of SU5402, dose-dependent inhibition of FGF2- but not the EGF-mediated activation of the ERK1/2 pathway was shown in PC12 cells (FIG. 8C). Together these studies provide evidence that NgR1 negatively regulates FGFR kinase signaling during hippocampal LTP at CA3-CA1 synapses.

OMgp Inhibits LTP at the Schaffer Collateral-CA1 Synapses in an NgR1-Dependent Manner

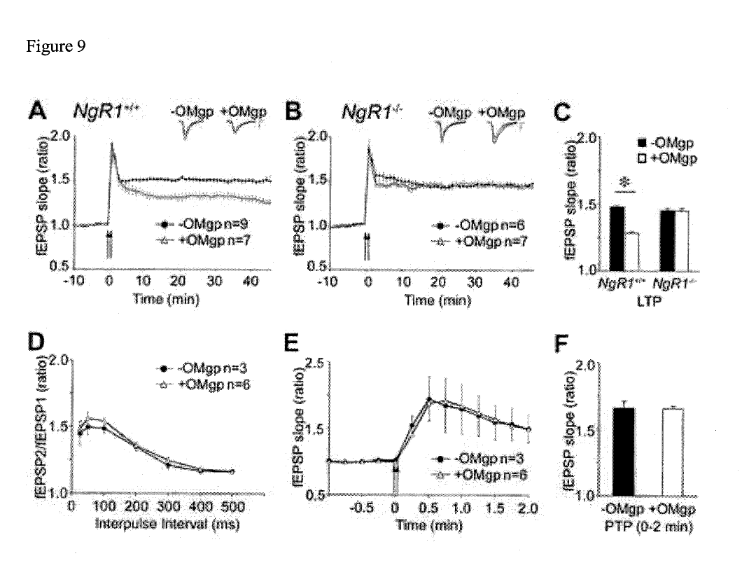

Whether NgR1 ligands other than FGF2 modulate LTP at the CA3-CA1 synapse was examined. In the mature nervous system, the myelin inhibitor OMgp is expressed by oligodendrocytes and is also abundantly found in cortical and hippocampal projection neurons (Habib et al., J. Neurochem. 70:1704-11, 1998). In the mature hippocampus, OMgp is found at synapses and localized to both pre- and postsynaptic sites. In wild-type hippocampal slices, focal application of soluble OMgp via the recording electrode results in a significant (p<0.001) attenuation of HFS-induced LTP at the Schaffer collateral [symbol] CA1 synapse (HFS; 100 Hz, 1 sec, separated by a 10 second interval). Compared to ACSF controls (mean fEPSP=148±2% of baseline, n=9 slices/7 animals), LTP in OMgp-treated slices consolidates at a significantly lower level (mean fEPSP=128±2%, n=7 slices/4 animals) and remains stable for at least one hour (FIG. 9A,C). In marked contrast to wild-type slices, NgR1 null hippocampal slices showed no attenuation of HFS-induced LTP in the presence of OMgp (mean fEPSP=145±1%, n=7 slices/4 animals) compared to ACSF controls (145±2%, n=6 slices/5 animals, p=0.994), indicating that in the mature hippocampus NgR1 functions as an OMgp receptor necessary for OMgp-mediated inhibition of LTP (FIG. 9B, C). Similar to studies with FGF2, assessment of PPF and PTP failed to support a presynaptic involvement of OMgp. In wild-type slices, no changes in PPF or PTP were observed in the presence of OMgp (FIG. 9D-F). Together, the results identify a new function for OMgp in regulating long-term synaptic plasticity and show that NgR1 serves as an obligatory receptor for OMgp-mediated inhibition of LTP at the Schaffer collateral-CA1 synapse.

NgR1 Mutant Mice Exhibit a Dendritic Spine Phenotype

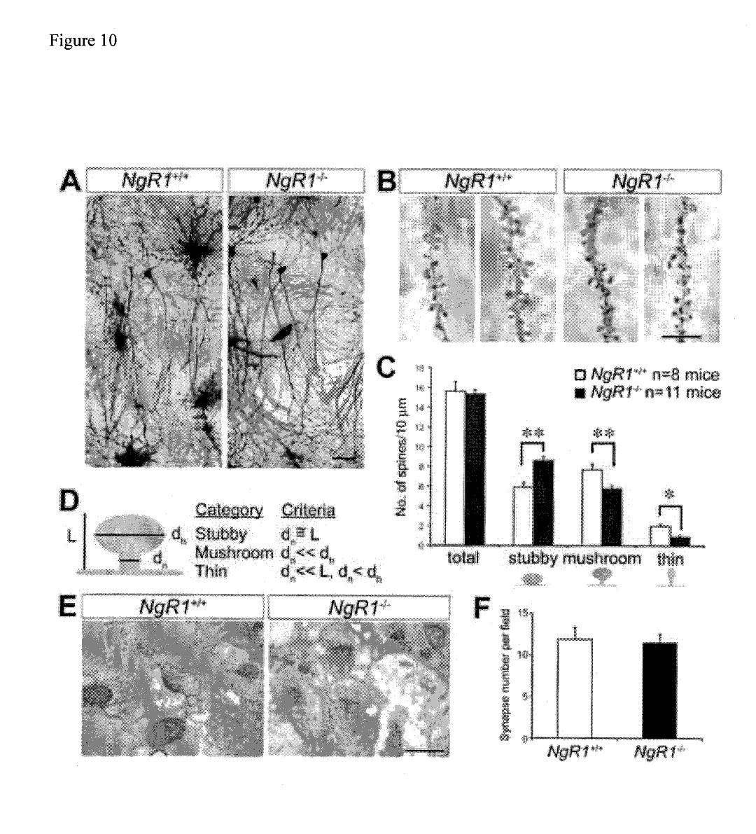

Investigations were performed to determine whether physiological NgR1 signaling regulates dendritic structure. The morphology of hippocampal neurons in adult wild-type and NgR1 mutants was assessed by Golgi staining. No apparent alterations in dendritic orientation or gross neuronal architecture of neocortical neurons and hippocampal CA1 pyramidal cells were observed in adult NgR1 mutants (FIG. 10A). During normal development, spines undergo morphological changes as they mature. In CA1 apical dendrites, for example, the density of stubby spines decreases with maturation, while that of mushroom-shaped spines increases (Grossman et al., Brain Res. 1084:158-64, 2006; Harris et al., J. Neurosci. 12:2685-2705, 1992). Golgi impregnation revealed no changes in dendritic spine density along secondary or tertiary branches of CA1 apical dendrites of wild-type (15.5 spines/10 μm; n=8 mice; 2356 spines) and NgR1 mutants (15.3 spines/10 μm n=11 mice; 5855 spines, p=0.51) (FIG. 10B,C). However, as the adult wild-type and NgR1 mutant dendritic spines were categorized into specific morphological spine categories (stubby, mushroom, or thin; see FIG. 10D for details), analysis of spine morphologies revealed a shift in distribution towards more stubby (p<0.001) and less mushroom shaped (p<0.001) and thin spines (p=0.04) in NgR1 mutants compared to wild-type controls (FIG. 10C,D; FIG. 15). Thus, rather than a change in spine density per dendritic segment, a shift in the frequency of spines with distinct shapes was detected. Consistent with this observation, ultrastructural analyses of the CA1 dendritic field revealed no significant changes (p=0.214) in synaptic density or shape between wild-type and NgR1 mutant mice (FIG. 10E,F). The increase in stubby spines is primarily at the expense of mushroom-shaped and, to a lesser extent, of thin spines, implying that NgR1 function is necessary for the proper development or maintenance of mushroom shaped spines. In sum, these studies show that NgR1 is an important regulator of neuronal structure in vivo.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

1. A polypeptide comprising a fragment of NgR1, wherein the NgR1 fragment has reduced FGF2 binding as compared to wild-type NgR1.

2. The polypeptide of claim 1, wherein the modified NgR1 fragment comprises a first amino acid sequence having at least about 80%, 90%, or 95% identity to SEQ ID NO:2.

3. The polypeptide of claim 2, wherein the modified NgR1 fragment further comprises a second amino acid sequence having at least about 80%, 90%, or 95% identity to SEQ ID NO:3.

4. (canceled)

5. (canceled)

6. (canceled)

7. (canceled)

8. The polypeptide of claim 1, wherein the modified NgR1 fragment comprises a first amino acid sequence comprising SEQ ID NO: 2 with up to twenty amino acid mutations.

9. The polypeptide of claim 8, wherein the modified NgR1 fragment further comprises a second amino acid sequence comprising SEQ ID NO: 3 with up to twenty amino acid mutations.

10. A nucleic acid encoding the polypeptide of claim 1 or a complement of the nucleic acid.

11. A vector comprising the nucleic acid of claim 10.

12. A cultured cell comprising the vector of claim 11.

13. A chimeric polypeptide comprising the fragment of claim 1 and a fragment of NgR2 or a fragment of an NgR2 variant, wherein the chimeric polypeptide comprises a ligand binding domain.

14. (canceled)

15. The chimeric polypeptide of claim 13, wherein the modified NgR2 fragment comprises an amino acid sequence having at least about 80%, 90%, or 95% identity to SEQ ID NO:5 or SEQ ID NO:6.

16. (canceled)

17. (canceled)

18. The chimeric polypeptide of claim 13, wherein the modified NgR2 fragment comprises SEQ ID NO: 5 or SEQ ID NO:6 with up to twenty amino acid mutations.

19. A nucleic acid encoding the polypeptide of claim 13 or a complement of the nucleic acid.

20. A vector comprising the nucleic acid of claim 19.

21. A cultured cell comprising the vector of claim 20.

22. A composition comprising
(a) the polypeptide of claim 1 and
(b) a pharmaceutically acceptable carrier or a culture medium.

23. A method of promoting neurite outgrowth comprising contacting a neuron with the composition of claim 22.

24. A method of promoting regeneration of the nervous system in a subject in need thereof comprising administering to the subject the composition of claim 22.

25. A composition comprising
(a) the NgR1 fragment of claim 1;
(b) a fragment of NgR2 or a modified fragment of NgR2; and
(c) FGF2.

26. The composition of claim 25, wherein the concentration of FGF2 is at least about 10 nanograms/ml, 50 nanograms/ml, or 100 nanograms/ml.

27. (canceled)

28. (canceled)

29. (canceled)

30. A method of promoting neurite outgrowth comprising contacting a neuron with the composition of claim 25.

31. A method of treating a central nervous system disease or disorder in a subject comprising administering to the subject the composition of claim 25.

32. A method of promoting activity-dependent synaptic strength comprising contacting a postsynaptic neuron with an agent that blocks NgR1 expression or NgR1 ligand binding.

33. (canceled)

34. (canceled)

35. The method of claim 32, wherein the agent is a fragment of NgR1.

36. The method of claim 32, wherein the agent is a soluble NgR1 or a variant thereof that binds OMgp.

37. The method of claim 32, wherein the agent is in siRNA.

38. (canceled)

39. (canceled)

40. A method of treating a subject with a neurodegenerative disease or condition comprising
(a) administering to the subject an agent that blocks NgR1 expression or NgR1 ligand binding and
(b) administering to the subject FGF2 or an agonist of FGF2.

41. The method of claim 39, wherein the neurodegenerative disease or condition is an injury to the central nervous system.

42. (canceled)

43. (canceled)

44. (canceled)

45. A chimeric polypeptide comprising the amino acid sequence of SEQ ID NO:2, the amino acid sequence of SEQ ID NO:6, and the amino acid of SEQ ID NO:3.
